(19) United States
(12) Patent Application Publication
(10) Pub. No.: US 2023/0149383 A1
(43) Pub. Date: May 18, 2023

(54) QUINOLINE COMPOUNDS FOR TREATING LUNG, LIVER, AND KIDNEY DISEASES, DISORDERS, OR CONDITIONS

(71) Applicant: Aldeyra Therapeutics, Inc., Lexington, MA (US)

(72) Inventors: Todd BRADY, Carlisle, MA (US); Adam BROCKMAN, Arlington, MA (US); Stephen Gitu MACHATHA, Wilmington, MA (US)

(21) Appl. No.: 17/918,047

(22) PCT Filed: Apr. 13, 2021
(86) PCT No.: PCT/US2021/027148

§ 371 Date: Oct. 10, 2022

Related U.S. Application Data
(60) Provisional application No. 63/027,713, filed on May 20, 2020.
(60) Provisional application No. 63/009,281, filed on Apr. 13, 2020.

(51) Int. Cl. A61K 31/47; A61P 11/00; A61P 1/16

(52) U.S. Cl. CPC A61K 31/47; A61P 11/00; A61P 1/16; C07B 2200/05

(57) ABSTRACT

The present invention relates to the use of a quinoline compound, or a pharmaceutically acceptable salt thereof, for treatment of a respiratory disease, disorder, or condition selected from chronic cough, pneumonia, and pulmonary sepsis, or an organ L disease, disorder, or condition selected from alcohol induced hepatitis, minimal change disease, and focal segmental glomerulosclerosis.

### CROSS-REFERENCE TO RELATED APPLICATIONS

The present application claims the benefit under 35 U.S.C. § 119(e) of U.S. Provisional Application Serial Nos. 63/027,713, filed on May 20, 2020; and 63/009,281, filed on Apr. 13, 2020; the entire contents of which are incorporated herein by reference.

### TECHNICAL FIELD

The present invention relates to methods of treating diseases, disorders or conditions affecting the lung, liver and kidney with the use of quinoline compounds.

### BACKGROUND

Chronic cough, pneumonia, and pulmonary sepsis are clinically distinct respiratory diseases, disorders, or conditions. Chronic cough is generally defined as cough lasting longer than 8 weeks and excluding cough with an underlying fever, such as from a bacterial or viral infection; chronic obstructive pulmonary disease (COPD) and other non-asthmatic pulmonary diseases; cancer of the lung or esophagus; pneumonia; interstitial lung disease; and obstructive sleep apnea. Pneumonia is an infection of the lungs by a pathogen, such as a bacteria, virus, or fungi. It is distinguished from Acute Respiratory Distress Syndrome, which can be caused by acute injury to the lung unrelated to infection by a pathogen. Pneumonia is usually diagnosed by a combination of clinical history, physical examination and/or laboratory tests, and clinical diagnosis from a chest X-ray (CXR), which can distinguish pneumonia from other respiratory tract infections. Pulmonary sepsis also affects the lungs but can arise from sepsis due to the sensitivity of the lungs and because sepsis can develop from infection of the lungs by a pathogen.

Atopic asthma is the most common form of asthma, affecting 70-90% of children and about 50% of adult sufferers. Exposure to environmental proteins called allergens is responsible for the characteristic symptoms. Allergens are ubiquitous. Knowledge of an individual's provoking triggers via a careful history may lead to successful avoidance measures. Where conventional treatment fails, immunomodulation may be considered in the most severe cases. An individual with atopic asthma will have mast cell-bound IgE molecules residing in his airways. Inhalation of the offending allergen leads to cross-linking of adjacent IgE molecules, causing mast cell activation and release of mediators including histamine and tryptase. This leads to an immediate or acute-phase asthmatic reaction, peaking at 15 minutes and resolving within an hour. Around 50% of asthmatics also experience a late-phase reaction at about six hours, due to a Th2 lymphocyte-mediated influx of inflammatory cells, eosinophils in particular, and further release of mediators.

Alcohol induced hepatitis, minimal change disease, and focal segmental glomerulosclerosis affect the liver or kidneys rather than the lungs. Alcohol induced hepatitis is attributed to chronic abuse of alcohol, and is characterized by injury to the liver. Defining characteristics include hyperbilirubinemia and levels of liver function markers aspartate aminotransferase (AST) and alanine aminotransferase (ALT). Minimal change disease and focal segmental glomerulosclerosis are diseases, disorders, or conditions affecting the kidney. Both minimal change disease and focal segmental glomerulosclerosis are within the broader disorder of nephrotic syndrome, and are characterized by proteinuria. Minimal change disease can progress into focal segmental glomerulosclerosis, where the latter involve injury and scarring to the kidney in a focal, segmental pattern.

Treatments for each of the diseases, disorders, or conditions are varied as their etiology. Steroids and some immune modulators are used to treat or alleviate the symptoms associated with these disorders. For example, corticortisteroids have been used to treat pneumonia, with mixed results (see, e.g., Stern et al., Cochrane Database Syst Rev., 2017; 2017(12):CD007720). Corticosteroids, such as prednisolone, have been used to treat minimal change disease and can lead to complete remission in over 80% of adults with the disease, with the duration of therapy lasting 4 weeks for about 50% of patients and 12 to 16 weeks of therapy for about 10% to 25% of patients (see, e.g., Hogan et al., J Amer Soc Nephrol., 2013; 24 (5):702-711). It is desirable to have other therapeutic agents having effectiveness for the spectrum of these specific but disparate disorders.

### SUMMARY

The present invention relates to use of quinoline compounds, and pharmaceutically acceptable salts thereof and compositions thereof, for treating respiratory disorders selected from chronic cough, atopic asthma, pneumonia, and pulmonary sepsis, and organ diseases selected from alcohol induced hepatitis, minimal change disease, and focal segmental glomerulosclerosis. In one aspect of the present invention, the compounds have general formula I.

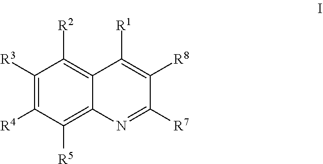

or a pharmaceutically acceptable salt thereof, wherein each variable is as defined herein.

In some embodiments, the compounds disclosed herein, or pharmaceutically acceptable salts thereof and compositions thereof, are useful for treating chronic cough.

In some embodiments, the compounds disclosed herein, or pharmaceutically acceptable salts thereof and compositions thereof, are useful for treating atopic asthma.

In some embodiments, the compounds disclosed herein, or pharmaceutically acceptable salts thereof and compositions thereof, are useful for treating pneumonia.

In some embodiments, the compounds disclosed herein, or pharmaceutically acceptable salts thereof and compositions thereof, are useful for treating pulmonary sepsis.

In some embodiments, the compounds disclosed herein, or pharmaceutically acceptable salts thereof and compositions thereof, are useful for treating alcohol induced hepatitis.

In some embodiments, the compounds disclosed herein, or pharmaceutically acceptable salts thereof and compositions thereof, are useful for treating minimal change disease.

In some embodiments, the compounds disclosed herein, or pharmaceutically acceptable salts thereof and compositions thereof, are useful for treating focal segmental glomerulosclerosis.

In some embodiments, the compounds disclosed herein, or pharmaceutically acceptable salts thereof and compositions thereof, are useful for treating allergic rhinitis.

In some embodiments, the compounds disclosed herein, or pharmaceutically acceptable salts thereof and compositions thereof, are useful for treating non-alcoholic fatty liver disease (NAFLD) or fatty liver disease.

In some embodiments, the compounds disclosed herein, or pharmaceutically acceptable salts thereof and compositions thereof, are useful for treating non-alcoholic steatohepatitis (NASH).

In some embodiments, a method of treating the above disorders, diseases or conditions comprise administering to a patient in need thereof an effective amount of a quinoline compound, or a pharmaceutically acceptable salt thereof, as disclosed herein.

In some embodiments, the compound or pharmaceutically acceptable salt thereof is administered systemically.

In some embodiments, the compound or pharmaceutically acceptable salt thereof is administered orally.

### BRIEF DESCRIPTION OF THE FIGURES

FIG. 1 shows a schematic of the study design for a Phase 2 trial to study the safety and efficacy of compound I-1 in subjects with mild asthma induced by the bronchial allergen challenge (BAC). MCT: Methacholine Challenge Test; BAC: Bronchial Allergen Challenge; FeNO: Fractional Exhaled Nitric Oxide; A: Compound I-1 600 mg PO bid for 7 (+3) Days; B: Placebo 600 mg PO bid for 7 (+3) Days.

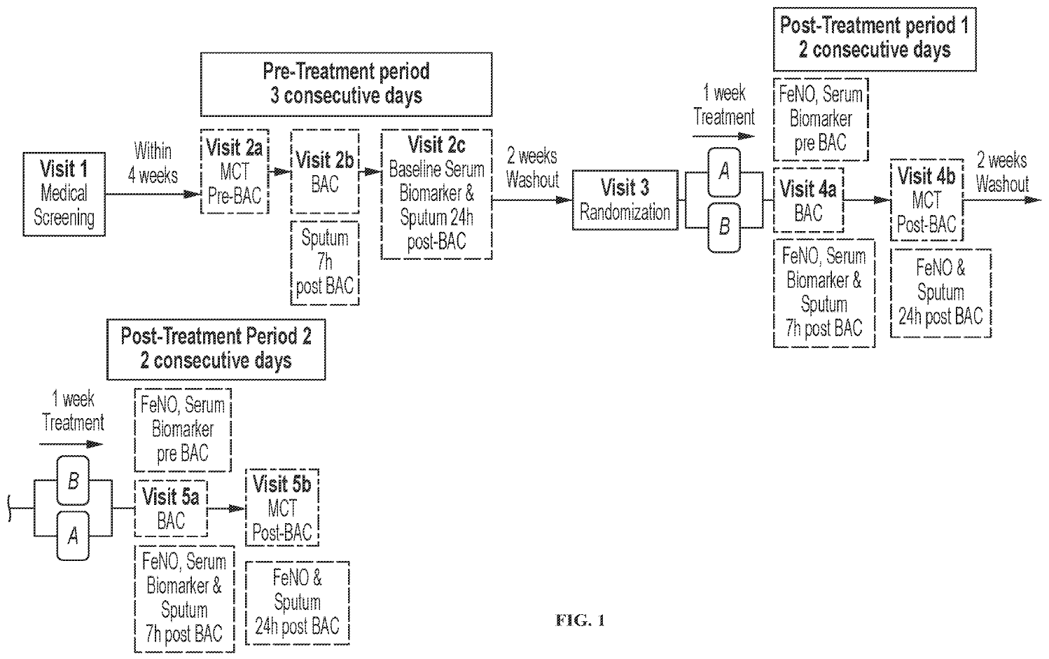

FIG. 2 shows the effect of compound I-1 (“ADX” in the bar graph) on triglyceride levels in human precision cut liver slices (PCLS) and on acetaldehyde (AA) levels. Levels were measured at 24 h, 48 h, and 72 h (left, middle, and right data bars, respectively).

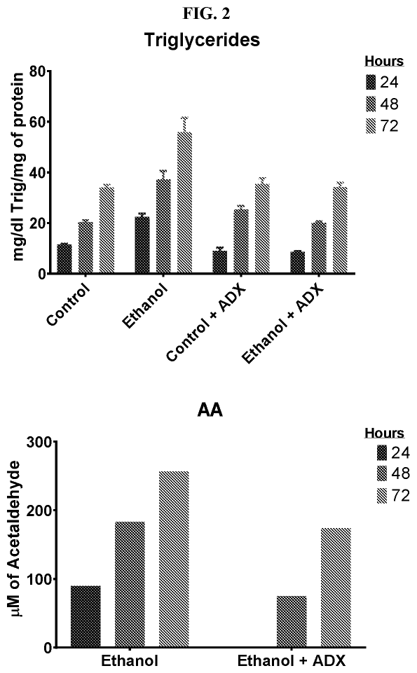

FIG. 3 shows the effect of compound I-1 (“ADX” in the bar graph) on ATP levels in human precision cut liver slices (PCLS) and on LDH levels. Levels were measured at 24 h, 48 h, and 72 h (left, middle, and right data bars, respectively).

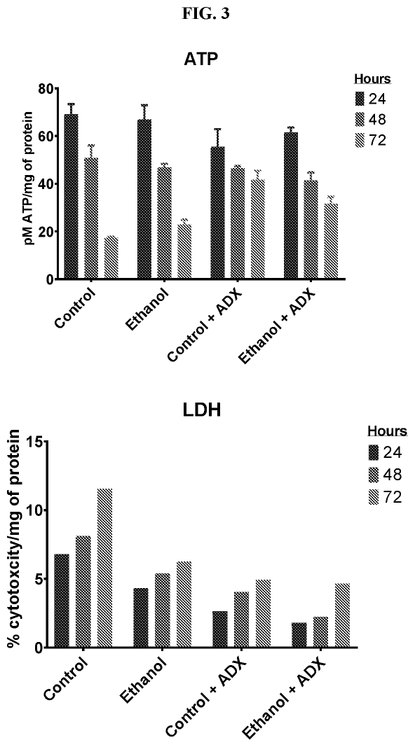

FIG. 4 shows the results of a 12-Week Choline Deficient (Amino Acid Defined) High Fat Diet in rats treated with compound I-1.

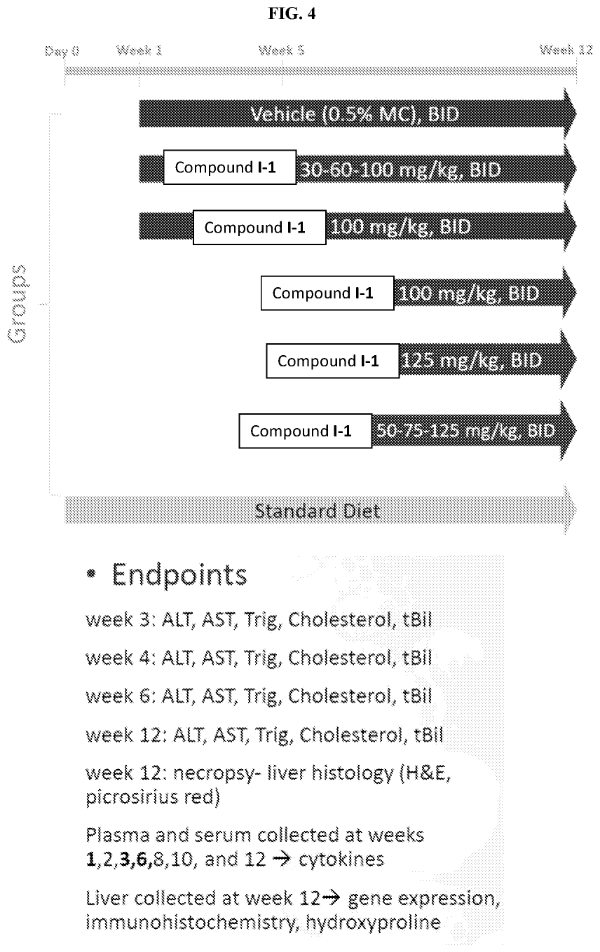

FIG. 5 shows food intake change, weight gain change from baseline, and levels of MIP, MCP, and RANTES cytokines for rats in the 12-week choline deficient high fat diet study with compound I-1.

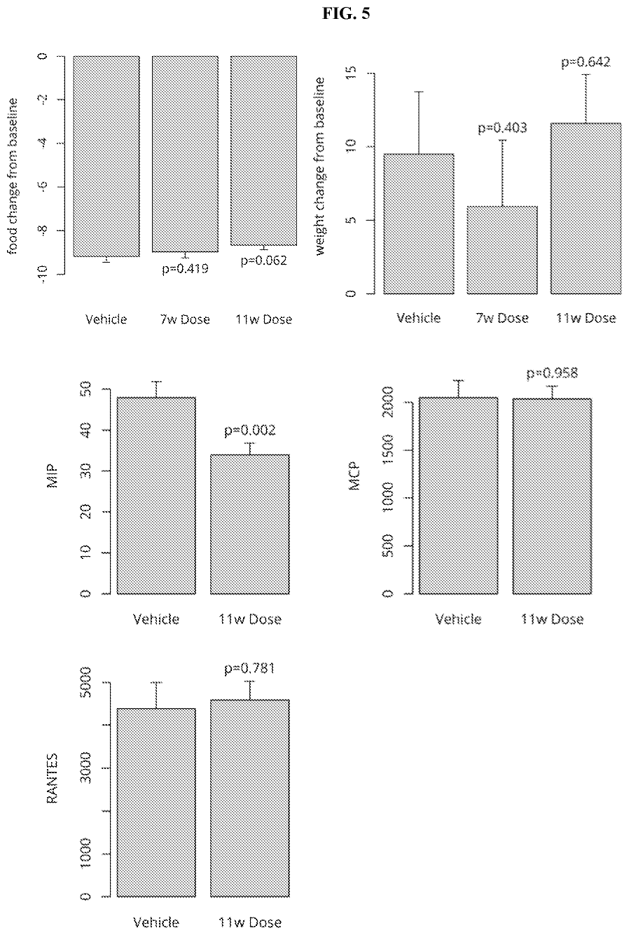

FIG. 6 shows cholesterol and triglyceride clinical chemistry for rats in the 12-week choline deficient high fat diet study with compound I-1. Cholesterol and triglycerides trended lower in the groups treated with compound I-1. Chol=cholesterol; trig=triglycerides; Bili=bilirubin.

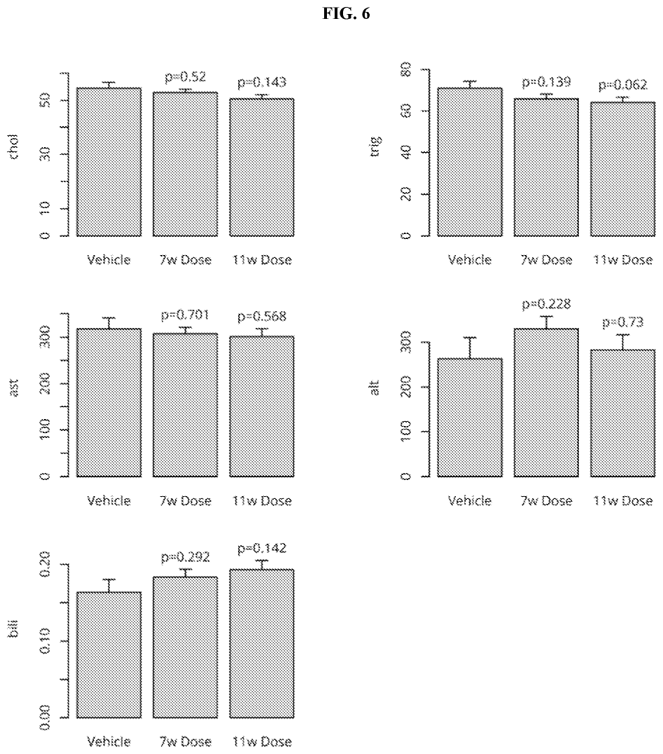

FIG. 7 shows histopathology results for rats in the 12-week choline deficient high fat diet study with compound I-1. Treatment reduced fibrosis and inflammation in I-1-treated groups. 7 w=7 week dose groups; 11 w=11 week dose groups.

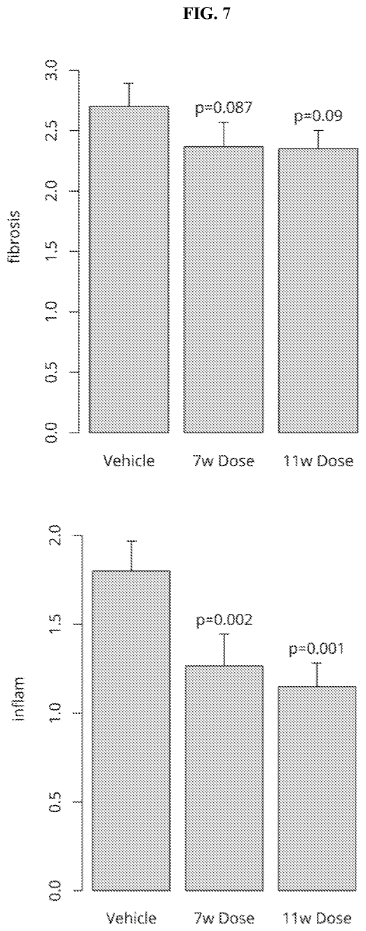

FIG. 8 shows NAS scores in a rat NAFLD model. 11 Week Groups (Day 84) NAS scores were lower in I-1-treated Groups. The NAS scoring system includes 4 semi-quantitative features: steatosis (0-3), lobular inflammation (0-2), hepatocellular ballooning (0-2), fibrosis (0-4).

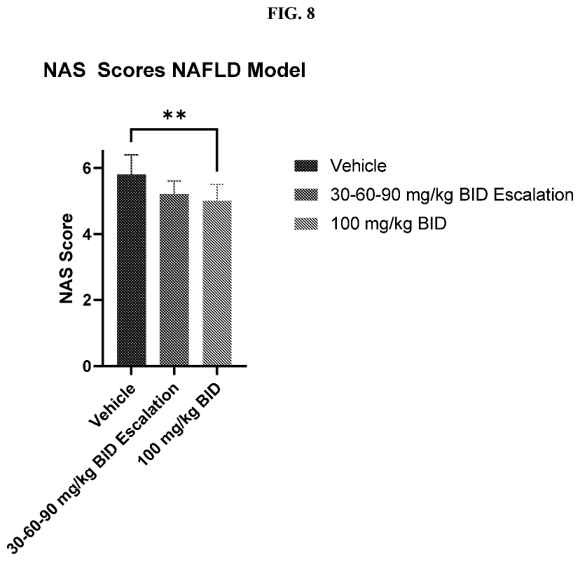

FIG. 9 shows the design and results for a STAM™ mouse study with compound I-1. The STAM™ model is a model that recapitulates the same disease progression as human NASH/HCC. In this model, male C57BL/6 mice aged two days are given a single dose of streptozotocin to reduce insulin secretory capacity. When the mice turn four weeks of age they start a high-fat diet feeding. This model has a background of late type 2 diabetes which progresses into fatty liver, NASH, fibrosis and consequently liver cancer (HCC). Compared to other NASH-HCC model mice, the disease progresses in a relatively short period of time, and liver cancer is developed in 100% of animals at 20 weeks of age. The model is widely used in NASH research, with more than 40 papers and 70 international conferences published using data from the STAM™ model so far. STAM™ model is able to reproduce many of the pathological features of human NASH. For example: ballooning degeneration of cells, a characteristic pathological feature of human NASH; burned-out NASH, in which lipid droplets decrease as fibrosis progresses; progression of fibrosis occurring around the central vein; a mild rise in ALT (a liver injury marker); increase in NASH markers such as CK-18; increase in human HCC markers such as glutamine synthase, glypican-3 and AFP have been observed. The data show that hepatic fibrosis and triglycerides are significantly reduced after treatment with compound I-1.

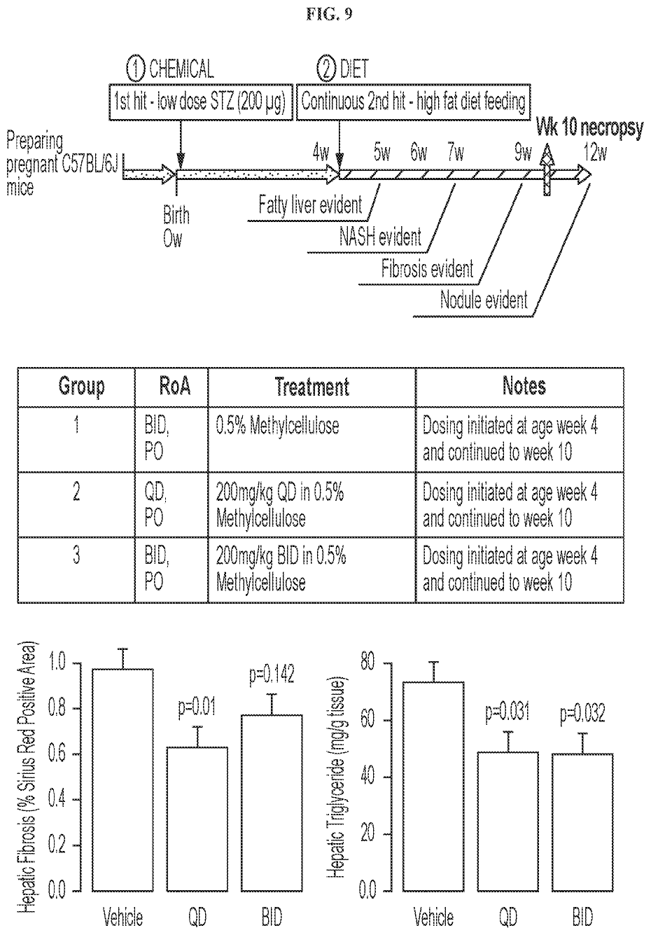

FIG. 10 shows statistically significant reduction of body weight gain in STAM™ rats treated with compound I-1 at 200 mg/kg QD or BID in methylcellulose.

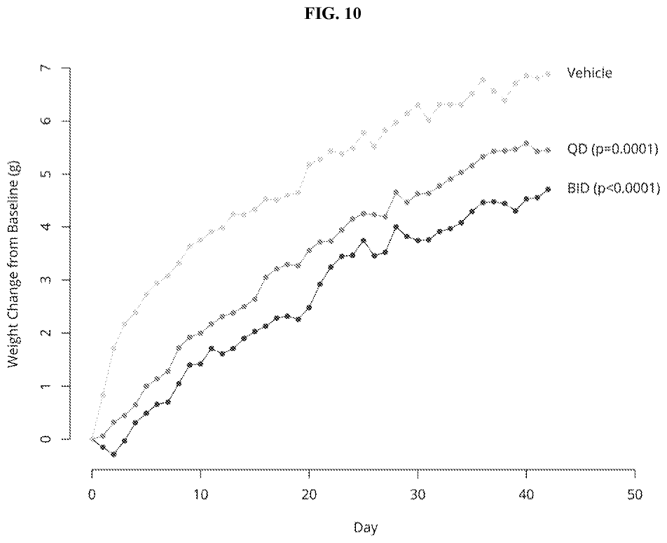

### DETAILED DESCRIPTION

### 1. General Description of Certain Aspects of the Invention

In some aspects, the present disclosure provides compounds, compositions, and methods for the treatment, amelioration, prevention, and/or reduction of a risk of a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis.

In some aspects, the present disclosure provides compounds, compositions, and methods for the treatment, amelioration, prevention, and/or reduction of a risk of a respiratory disease, disorder, or condition selected from allergic rhinitis, or an organ disease, disorder, or condition selected from NAFLD, fatty liver disease, and NASH.

In another aspect, the present disclosure provides compounds, compositions, and methods for the treatment, amelioration, prevention, and/or reduction of a risk of atopic asthma.

In one aspect, the present invention provides a method of treating a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, the method comprising administering to a patient in need thereof an effective amount of a compound of Formula I:

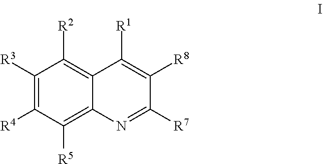

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

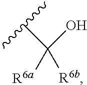

wherein one of R¹, R⁷, and R⁸ is —NH₂ and one of R¹ R⁷, and R⁸ is

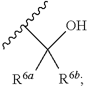

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In one aspect, the present invention provides a method of treating a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, comprising administering to a patient in need thereof an effective amount of a compound of formula II:

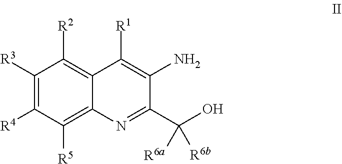

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

### 2. Definitions

Compounds of the present invention include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁-4 straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments, the term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. In certain embodiments of the compounds, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned for the compounds herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)⁰⁻¹Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)⁰⁻¹Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}2, —NO₂, —SiR^{●}3, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)⁰⁻¹Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁-6 aliphatic which may be substituted as defined below, and an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)⁰⁻¹Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁-6 aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)⁰⁻¹Ph, or a 5- to 6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

### 3. Detailed Description of Embodiments

The compounds described herein are quinoline compounds that have aldehyde trapping activity, and have been described for use in treating disorders and diseases associated with the effects of toxic aldehydes. See, e.g., PCT patent publication WO2006127945, WO2014116836, WO2017035077, and WO2017035082, each of which is hereby incorporated by reference. Synthesis of the compounds herein are described in PCT publications WO2006127945, WO2017035082, and WO2018039192; and U.S. patent application publication US 2013/0190500, each of which is hereby incorporated by reference.

In addition, the disclosures of the following patent applications are hereby incorporated by reference: WO 2019/075136, filed Oct. 10, 2018; and PCT/US2021/023884, filed Mar. 24, 2021. These applications provide additional disclosure related to the quinoline compounds described herein, including their use in treating certain diseases.

As described in the present disclosure, certain quinoline compounds are useful in treating a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis. In some embodiments, the respiratory disease, disorder, or condition is atopic asthma. In some aspects, the present disclosure provides compounds, compositions, and methods for the treatment, amelioration, prevention, and/or reduction of a risk of a respiratory disease, disorder, or condition selected from allergic rhinitis, or an organ disease, disorder, or condition selected from NAFLD, fatty liver disease, and NASH.

Accordingly, in one aspect, the present disclosure provides a method of treating a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, the method comprising administering to a patient in need thereof an effective amount of a compound of Formula I:

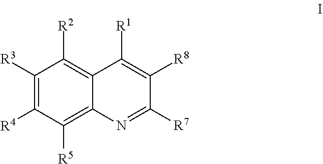

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

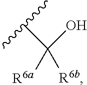

wherein one of R¹, R⁷, and R⁸ is —NH₂ and one of R¹, R⁷, and R⁸ is

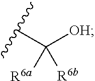

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁-4 aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In another aspect, the present disclosure provides a method of treating a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, the method comprising administering to a patient in need thereof an effective amount of a compound of Formula II:

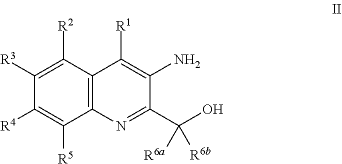

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

In another aspect, the present disclosure provides a method for treating, ameliorating, preventing, and/or reducing a risk of atopic asthma, comprising administering to a patient in need thereof an effective amount of a compound of Formula I or II; or a pharmaceutically acceptable salt thereof.

The following embodiments are applicable to Formula I.

In some embodiments of Formula I, R^{6a} is C₁₋₄ aliphatic. In some embodiments, R^{6a} is C₁₋₄ aliphatic optionally substituted with 1, 2, or 3 deuterium atoms. In some embodiments, R^{6a} is C₁₋₄ aliphatic optionally substituted with 1, 2, or 3 halogen atoms.

In some embodiments of formula I, R^{6a} is C₁₋₄ alkyl. In some embodiments, R^{6a} is C₁₋₄ alkyl optionally substituted with 1, 2, or 3 deuterium or halogen atoms. In some embodiments, R^{6a} is C₁₋₄ alkyl optionally substituted with 1, 2, or 3 halogen atoms. In some embodiments, R^{6a} is methyl or ethyl optionally substituted with 1, 2, or 3 halogen atoms. In some embodiments, R^{6a} is methyl.

As defined generally above, R^{6b} is C₁₋₄ aliphatic optionally substituted with 1, 2, or 3 deuterium or halogen atoms.

In some embodiments of formula I, R^{6b} is C₁₋₄ aliphatic. In some embodiments, R^{6b} is C₁₋₄ aliphatic optionally substituted with 1, 2, or 3 deuterium atoms. In some embodiments, R^{6b} is C₁₋₄ aliphatic optionally substituted with 1, 2, or 3 halogen atoms.

In some embodiments of formula I, R^{6b} is C₁₋₄ alkyl. In some embodiments, R^{6b} is C₁₋₄ alkyl optionally substituted with 1, 2, or 3 deuterium or halogen atoms. In some embodiments, R^{6b} is C₁₋₄ alkyl optionally substituted with 1, 2, or 3 halogen atoms. In some embodiments, R^{6b} is methyl or ethyl optionally substituted with 1, 2, or 3 halogen atoms. In some embodiments, R^{6b} is methyl.

As defined generally above, in some embodiments, R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur.

In some embodiments of Formula I, R^{6a} and R^{6b}, taken together with the carbon atom to which they are attached, form a 3- to 8-membered cycloalkyl. In some embodiments, R^{6a} and R^{6b}, taken together with the carbon atom to which they are attached, form a 3- to 8-membered heterocyclyl ring containing 1-2 heteroatoms selected from nitrogen, oxygen, and sulfur.

In some embodiments of Formula I, R^{6a} and R^{6b}, taken together with the carbon atom to which they are attached, form a cyclopropyl, cyclobutyl, or cyclopentyl ring. In some embodiments, R^{6a} and R^{6b}, taken together with the carbon atom to which they are attached, form an oxirane, oxetane, tetrahydrofuran, or aziridine.

In some embodiments of Formula I, the —NH₂ on one of R¹, R⁷, and R⁸ and the carbinol on the other of R¹, R⁷, and R⁸ are on adjacent carbon atoms of the pyridine moiety.

In some embodiments, the compound is a compound of Formula I-a, I-b, or I-c:

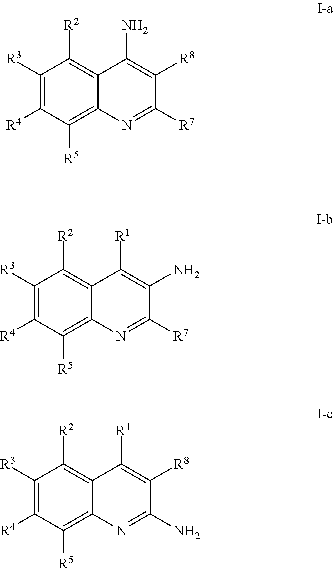

or a pharmaceutically acceptable salt thereof, wherein:
each of R¹, R⁷, and R⁸ when present is independently H, D, halogen, —CN, —OR, —SR, optionally substituted C₁₋₆ aliphatic, or

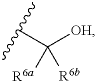

wherein one of R¹, R⁷, and R⁸ is

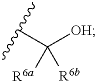

and
R², R³, R⁴, R⁵, R^{6a}, R^{6b}, R⁷, R⁸, and R are as defined for formula I.

In some embodiments, the compound for use in the method is a compound of formula I-d, I-e, I-f or I-g:

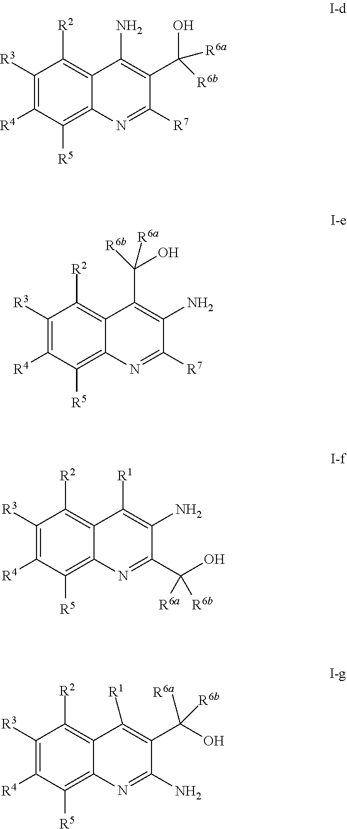

or a pharmaceutically acceptable salt thereof, wherein;
R¹ and R⁷ is independently H, D, halogen, —CN, —OR, —SR, optionally substituted C₁-6 aliphatic; and
R², R³, R⁴, R⁵, R^{6a}, R^{6b}, R⁷, R⁸, and R are as defined for Formula I.

The following embodiments are applicable to Formula II.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, or halogen.

In some embodiments, R³ is H. In some embodiments, R³ is D. In some embodiments, R³ is halogen. In some embodiments, R³ is Cl. In some embodiments, R³ is Br.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R^{6a} is C₁₋₄ aliphatic optionally substituted with 1, 2, or 3 deuterium or halogen atoms.

In some embodiments, R^{6a} is C₁₋₄ aliphatic substituted with 1, 2, or 3 deuterium or halogen atoms. In some embodiments, R^{6a} is C₁₋₄ aliphatic. In some embodiments, R^{6a} is C₁₋₄ alkyl. In some embodiments, R^{6a} is methyl, ethyl, n-propyl, or isopropyl. In some embodiments, R^{6a} is methyl.

As defined generally above, R^{6b} is C₁₋₄ aliphatic optionally substituted with 1, 2, or 3 deuterium or halogen atoms.

In some embodiments, R^{6b} is C₁₋₄ aliphatic substituted with 1, 2, or 3 deuterium or halogen atoms. In some embodiments, R^{6b} is C₁₋₄ aliphatic. In some embodiments, R^{6b} is C₁₋₄ alkyl. In some embodiments, R^{6b} is C₁₋₄ alkyl optionally substituted with 1, 2, or 3 fluorine atoms. In some embodiments, R^{6b} is methyl, ethyl, n-propyl, or isopropyl. In some embodiments, R^{6b} is methyl.

In some embodiments, R^{6a} and R^{6b} are methyl or ethyl. In some embodiments, R^{6a} and R^{6b} are methyl. In some embodiments, R^{6a} and R^{6b} are —CD₃.

In some embodiments, the compound is of Formula II-a:

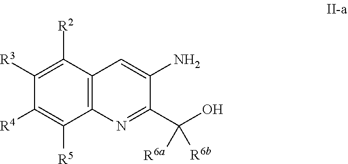

or a pharmaceutically acceptable salt thereof, wherein:
each of R², R³, R⁴, R⁵, R^{6a}, and R^{6b} is as defined as provided above and described in embodiments herein, both singly and in combination.

In some embodiments, the compound is of Formula II-b:

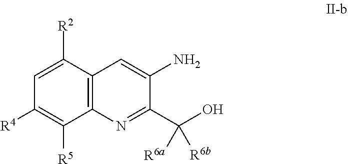

or a pharmaceutically acceptable salt thereof, wherein:
each of R², R⁴, R⁵, R^{6a}, and R^{6b} is as defined as provided above and described in embodiments herein, both singly and in combination.

In some embodiments, the compound is of any one of Formulae II-c, II-d, II-e, or II-f:

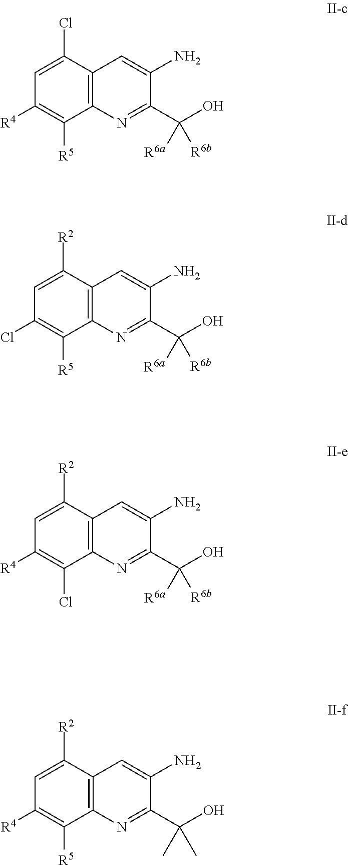

or a pharmaceutically acceptable salt thereof, wherein:
each of R², R⁴, R⁵, R^{6a}, and R^{6b} is as defined as provided above and described in embodiments herein, both singly and in combination.

In some embodiments, the compound is of Formula II-g:

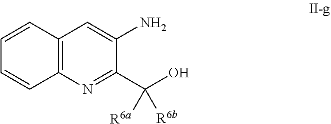

or a pharmaceutically acceptable salt thereof, wherein:
each of R^{6a} and R^{6b} is as defined as provided above and described in embodiments herein, both singly and in combination.

In some embodiments, a disclosed method comprises administering a compound selected from one depicted in Table 1, below.

TABLE 1
Representative Compounds

| | |
|---|---|
| 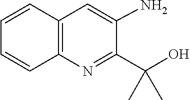 | I-1 |
| 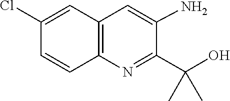 [SMILES: CC(C)(O)c1nc2ccc(Cl)cc2cc1N] | I-2 |
| 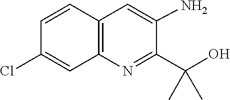 [SMILES: CC(C)(O)c1nc2cc(Cl)ccc2cc1N] | I-3 |
| 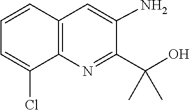 [SMILES: CC(C)(O)c1nc2c(Cl)cccc2cc1N] | I-4 |
| 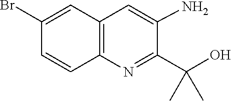 [SMILES: CC(C)(O)c1nc2ccc(Br)cc2cc1N] | I-5 |
| 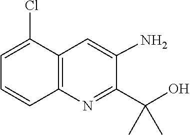 [SMILES: CC(C)(O)c1nc2cccc(Cl)c2cc1N] | I-6 |
| 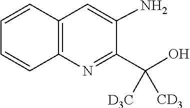 [SMILES: [2H]C([2H])([2H])C(O)(c1nc2ccccc2cc1N)C([2H])([2H])[2H]] | I-7 |
| 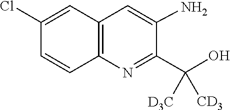 [SMILES: [2H]C([2H])([2H])C(O)(c1nc2ccc(Cl)cc2cc1N)C([2H])([2H])[2H]] | I-8 |
| 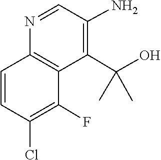 [SMILES: CC(C)(O)c1c(N)cnc2ccc(Cl)c(F)c12] | I-9 |
| 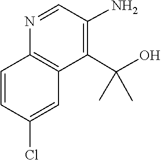 [SMILES: CC(C)(O)c1c(N)cnc2ccc(Cl)cc12] | I-10 |
| 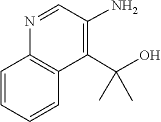 | I-11 |
| 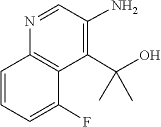 [SMILES: CC(C)(O)c1c(N)cnc2cccc(F)c12] | I-12 |
| 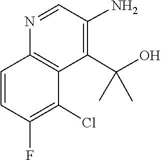 [SMILES: CC(C)(O)c1c(N)cnc2ccc(F)c(Cl)c12] | I-13 |
| 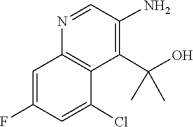 [SMILES: CC(C)(O)c1c(N)cnc2cc(F)cc(Cl)c12] | I-14 |
| 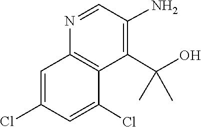 [SMILES: CC(C)(O)c1c(N)cnc2cc(Cl)cc(Cl)c12] | I-15 |
| 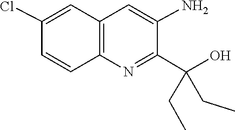 [SMILES: CCC(O)(CC)c1nc2ccc(Cl)cc2cc1N] | I-16 |
| 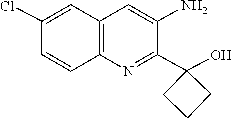 [SMILES: Nc1cc2cc(Cl)ccc2nc1C1(O)CCC1] | I-17 |
| 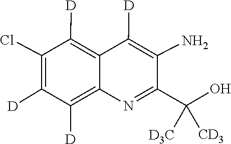 [SMILES: [2H]c1c(Cl)c([2H])c2c([2H])c(N)c(C(O)(C([2H])([2H])[2H])C([2H])([2H])[2H])nc2c1[2H]] | X-1 |
| 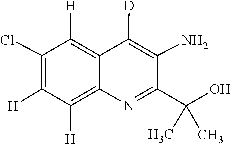 [SMILES: [2H]c1c(N)c(C(C)(C)O)nc2ccc(Cl)cc12] | X-2 |
| 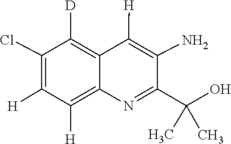 [SMILES: [2H]c1c(Cl)ccc2nc(C(C)(C)O)c(N)cc12] | X-3 |
| 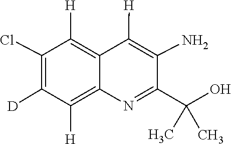 [SMILES: [2H]c1cc2nc(C(C)(C)O)c(N)cc2cc1Cl] | X-4 |
| 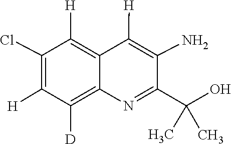 [SMILES: [2H]c1cc(Cl)cc2cc(N)c(C(C)(C)O)nc12] | X-5 |
| 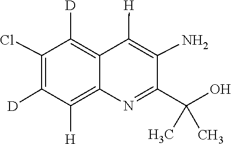 [SMILES: [2H]c1cc2nc(C(C)(C)O)c(N)cc2c([2H])c1Cl] | X-6 |
| 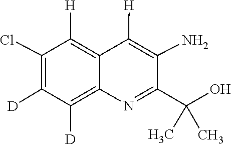 [SMILES: [2H]c1c(Cl)cc2cc(N)c(C(C)(C)O)nc2c1[2H]] | X-7 |
| 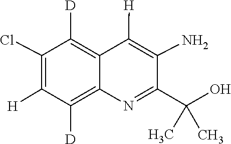 [SMILES: [2H]c1c(Cl)cc([2H])c2nc(C(C)(C)O)c(N)cc12] | X-8 |
| 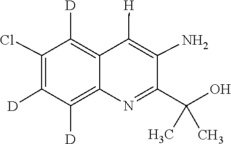 [SMILES: [2H]c1c(Cl)c([2H])c2cc(N)c(C(C)(C)O)nc2c1[2H]] | X-9 |
| 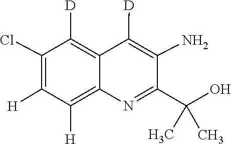 [SMILES: [2H]c1c(Cl)ccc2nc(C(C)(C)O)c(N)c([2H])c12] | X-10 |
| 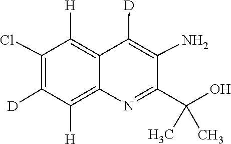 [SMILES: [2H]c1cc2nc(C(C)(C)O)c(N)c([2H])c2cc1Cl] | X-11 |
| 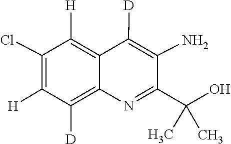 [SMILES: [2H]c1c(N)c(C(C)(C)O)nc2c([2H])cc(Cl)cc12] | X-12 |
| 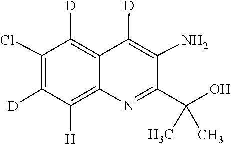 [SMILES: [2H]c1cc2nc(C(C)(C)O)c(N)c([2H])c2c([2H])c1Cl] | X-13 |
| 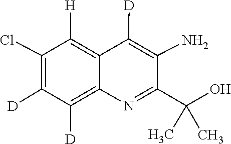 [SMILES: [2H]c1c(Cl)cc2c([2H])c(N)c(C(C)(C)O)nc2c1[2H]] | X-14 |
| 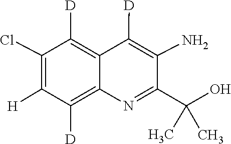 [SMILES: [2H]c1cc(Cl)c([2H])c2c([2H])c(N)c(C(C)(C)O)nc12] | X-15 |
| 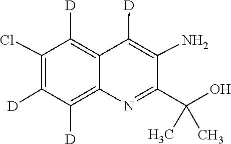 [SMILES: [2H]c1c(Cl)c([2H])c2c([2H])c(N)c(C(C)(C)O)nc2c1[2H]] | X-16 |
| 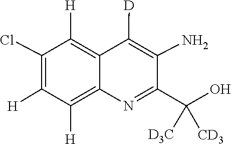 [SMILES: [2H]c1c(N)c(C(O)(C([2H])([2H])[2H])C([2H])([2H])[2H])nc2ccc(Cl)cc12] | X-17 |
| 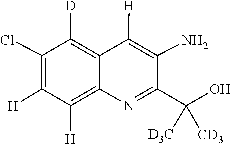 [SMILES: [2H]c1c(Cl)ccc2nc(C(O)(C([2H])([2H])[2H])C([2H])([2H])[2H])c(N)cc12] | X-18 |
| 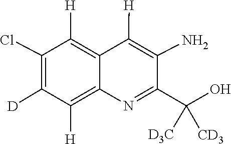 [SMILES: [2H]c1cc2nc(C(O)(C([2H])([2H])[2H])C([2H])([2H])[2H])c(N)cc2cc1Cl] | X-19 |
| 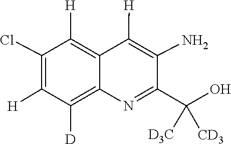 [SMILES: [2H]c1cc(Cl)cc2cc(N)c(C(O)(C([2H])([2H])[2H])C([2H])([2H])[2H])nc12] | X-20 |
| 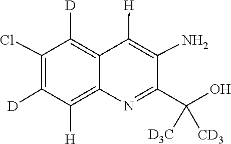 [SMILES: [2H]c1cc2nc(C(O)(C([2H])([2H])[2H])C([2H])([2H])[2H])c(N)cc2c([2H])c1Cl] | X-21 |
| 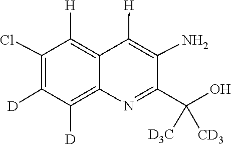 [SMILES: [2H]c1c(Cl)cc2cc(N)c(C(O)(C([2H])([2H])[2H])C([2H])([2H])[2H])nc2c1[2H]] | X-22 |
| 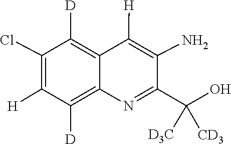 [SMILES: [2H]c1c(Cl)cc([2H])c2nc(C(O)(C([2H])([2H])[2H])C([2H])([2H])[2H])c(N)cc12] | X-23 |
| 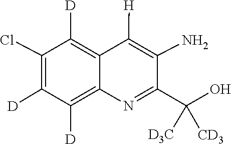 [SMILES: [2H]c1c(Cl)c([2H])c2cc(N)c(C(O)(C([2H])([2H])[2H])C([2H])([2H])[2H])nc2c1[2H]] | X-24 |
| 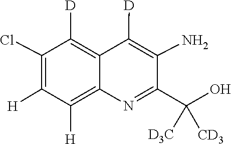 [SMILES: [2H]c1c(Cl)ccc2nc(C(O)(C([2H])([2H])[2H])C([2H])([2H])[2H])c(N)c([2H])c12] | X-25 |
| 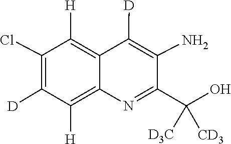 [SMILES: [2H]c1cc2nc(C(O)(C([2H])([2H])[2H])C([2H])([2H])[2H])c(N)c([2H])c2cc1Cl] | X-26 |
| 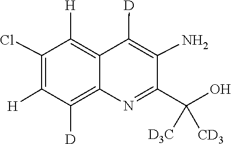 [SMILES: [2H]c1c(N)c(C(O)(C([2H])([2H])[2H])C([2H])([2H])[2H])nc2c([2H])cc(Cl)cc12] | X-27 |
| 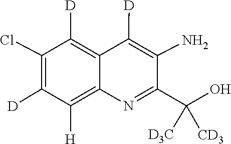 [SMILES: [2H]c1cc2nc(C(O)(C([2H])([2H])[2H])C([2H])([2H])[2H])c(N)c([2H])c2c([2H])c1Cl] | X-28 |
| 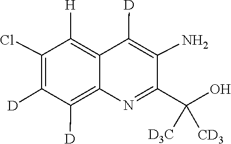 [SMILES: [2H]c1c(Cl)cc2c([2H])c(N)c(C(O)(C([2H])([2H])[2H])C([2H])([2H])[2H])nc2c1[2H]] | X-29 |
| 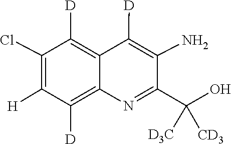 [SMILES: [2H]c1cc(Cl)c([2H])c2c([2H])c(N)c(C(O)(C([2H])([2H])[2H])C([2H])([2H])[2H])nc12] | X-30 |

In some embodiments, the present disclosure provides a compound depicted in Table 1, above, or a pharmaceutically acceptable salt thereof, for use in a method of treatment described herein.

In some aspects, the present disclosure provides a compound described herein or pharmaceutically acceptable salt thereof for use in a method for the treatment, amelioration, prevention, and/or reduction of allergic rhinitis. In some aspects, the present disclosure provides a compound described herein or pharmaceutically acceptable salt thereof for use in a method for the treatment, amelioration, prevention, and/or reduction of an organ disease, disorder, or condition selected from NAFLD, fatty liver disease, and NASH. In some embodiments, the compound is a compound depicted in Table 1, above, such as compound I-1.

In some embodiments, the present disclosure provides any compound described above and herein, or a pharmaceutically acceptable salt thereof, for use in a disclosed method of treatment. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

In some embodiments, the compounds described herein are used for the treatment, prevention, and/or reduction of a risk of respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis.

In some embodiments, the compounds described herein are used for the treatment, prevention, and/or reduction of a risk of atopic asthma. In some embodiments, the atopic (or allergic) asthma is triggered by an allergen such as an indoor, outdoor, or occupational allergen, including pollen, dust, an animal (e.g., cat dander or dog hair), or dust mites. In some embodiments, the atopic asthma patient also has another condition selected from seasonal allergies, eczema, and a food allergy.

As noted above, in one aspect the present disclosure provides a method of treating, preventing, and/or reducing of a risk of respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, the method comprising administering an effective amount of a compound described herein.

In some embodiments, the present disclosure provides use of the compound described herein in the manufacture of a medicament for the treatment, prevention, and/or reduction of a risk of respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis.

In some embodiments, the compound is any one of the exemplary compounds of Table 1.

In some embodiments, the compound for use in treating, preventing, and/or reducing the risk of a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, is a compound of Formula II-g or a pharmaceutically acceptable salt thereof.

In some embodiments, the compound for use in treating, preventing, and/or reducing risk of a respiratory disease, disorder, or condition selected from chronic cough, pneumonia, and pulmonary sepsis, or an organ disease, disorder, or condition selected from alcohol induced hepatitis, minimal change disease, and focal segmental glomerulosclerosis, is compound I-1 or I-2, or a pharmaceutically acceptable salt thereof.

In some embodiments, the focal segmental glomerulosclerosis (FSGS) is primary FSGS. Many people diagnosed with FSGS have no known cause for their condition. This is called primary (idiopathic) FSGS.

In some embodiments, the focal segmental glomerulosclerosis (FSGS) is secondary FSGS. Several factors, such as infection, drug toxicity, diseases such as diabetes or sickle cell disease, obesity, and even other kidney diseases can cause secondary FSGS. Controlling or treating the underlying cause often halts ongoing kidney damage and might lead to improved kidney function over time.

In some embodiments, the focal segmental glomerulosclerosis (FSGS) is genetic (also called familial) FSGS. This rare form of FSGS is caused by genetic mutations. Familial FSGS can also occur when neither parent has the disease, but each carries one copy of an abnormal gene that can be passed on to the next generation.

In some embodiments, a method of the disclosure is directed to treatment of chronic cough. In some embodiments, a method of treating or reducing the risk of chronic cough comprises administering to a patient in need thereof an effective amount of a compound disclosed herein. Generally, chronic cough is characterized as cough lasting greater than 8 weeks duration (see, e.g., Irwin et al., Chest, 2018; 153(1):196-209; Morice, A. H., European Respiratory J., 2004; 24:481-492). Chronic cough can be triggered by and/or arise from different underlying causes, such as asthma, gastroesophageal reflux disease (GERD), non-asthmatic eosinophilic bronchitis (NAEB), and upper airway cough syndrome, otherwise known as postnasal drip syndrome. A differential diagnosis of chronic cough excludes cough accompanied by fever, such as from a bacterial or viral infection; chronic obstructive pulmonary disease (COPD) and other non-asthmatic pulmonary diseases; cancer of the lung or esophagus; pneumonia; interstitial lung disease; and obstructive sleep apnea (see, e.g., Perotin et al., Ther Clin Risk Manag, 2018: 14:1041-1051).

In some embodiments, the chronic cough for treatment is associated with upper airway cough syndrome.

In some embodiments, the chronic cough for treatment is associated with gastroesophageal reflux disease or laryngopharyngeal reflux disease.

In some embodiments, the chronic cough for treatment is associated with asthma.

In some embodiments, the chronic cough for treatment is associated with non-asthmatic eosinophilic bronchitis.

In some embodiments, the patient treated has a history of one or more of the following: treatment with angiotensin-converting enzyme (ACE) inhibitor, smoking, asthma, exposure to environmental respiratory irritants, and bronchitis.

In some embodiments, a method of the disclosure is directed to treatment of pneumonia. In some embodiments, the pneumonia is not associated or concurrent with acute respiratory distress syndrome (ARDS).

In some embodiments, the patient treated has pneumonia, wherein the pneumonia has a differential diagnosis from eosinophilic pneumonia (i.e., the pneumonia is not associated with eosinophilic pneumonia).

In some embodiments, the pneumonia treated is community-acquired pneumonia.

In some embodiments, the pneumonia treated is nocosomial pneumonia.

In some embodiments, the pneumonia treated is bacterial pneumonia or viral pneumonia.

In some embodiments, the patient treated is diagnosed with a bacterial infection by, among others, Streptococcus pneumoniae, Haemophilus influenzae, S. aureus, Group A streptococci, Moraxella catarrhalis, Klebsiella pneumoniae, Pseudomonas aeruginosa, Legionella spp, Mycoplasma pneumoniae, Chlamydia pneumoniae, or C. psittaci.

In some embodiments, the patient treated is diagnosed with a viral infection by influenza virus (e.g., influenza A or influenza B), respiratory syncytial virus (RSV), parainfluenza, metapneumovirus, coronavirus, rhinovirus, hantavirus, or adenovirus.

In some embodiments, the pneumonia treated is lobar pneumonia.

In some embodiments, the pneumonia treated is upper, middle or lower lobe pneumonia.

In some embodiments, the pneumonia treated is focal pneumonia, alveolar pneumonia, or interstitial pneumonia.

In some embodiments, the pneumonia treated is bronchial pneumonia.

In some embodiments, a method of the disclosure is directed to treatment of pulmonary sepsis or sepsis-induced lung injury. In some embodiments, a method of treating or reducing the risk of pulmonary sepsis or sepsis-induced lung injury comprises administering to a patient in need thereof an effective amount of a compound disclosed herein. Generally, pulmonary sepsis or sepsis induced lung injury is characterized as lung injury arising from sepsis. The lung is the organ most often affected by sepsis primarily because pneumonia is often the starting point of the septic process, and disseminated infectious process is associated with a systemic inflammatory response (SIRS) in which the first organ to be affected is usually the lung.

In some embodiments, the pulmonary sepsis or sepsis induced lung injury treated is without (i.e., not associated with) acute respiratory distress syndrome (ARDS).

In some embodiments, a method of the disclosure is directed to treatment of alcohol induced hepatitis. In some embodiments, a method of treating or reducing the risk of alcohol induced hepatitis comprises administering to a patient in need thereof an effective amount of a compound disclosed herein. Generally, alcohol induced hepatitis is liver injury and associated inflammatory condition arising from chronic alcohol abuse. A prominent feature or marker for the disease is hyperbilirubinemia. In some embodiments, alcohol induced hepatitis is distinguished from cirrhosis in that the former appears reversible while the latter is a permanent injury to the liver.

In some embodiments, the alcohol induced hepatitis is without cirrhosis (i.e., not accompanied by cirrhosis).

In some embodiments, the patient treated for alcohol induced hepatitis is determined to have elevated levels of aspartate aminotransferase (AST) and/or alanine aminotransferase (ALT) as compared to levels in a control group not afflicted with alcohol induced hepatitis.

In some embodiments, the levels of AST in the control group (i.e., without alcohol induced hepatitis) is about 8 to 48 IU/L and the levels of ALT in the control group is about 7 to 55 IU/L.

In some embodiments, the patient treated has a AST:ALT ratio of greater than 2:1.

This ratio is characteristic in patients with alcoholic liver disease. Patients with a history of alcohol abuse but no significant alcoholic hepatitis or cirrhosis of the liver usually have an AST/ALT ratio less than 1.0.

In some embodiments, a method of the disclosure is directed to treatment of minimal change disease, sometimes referred to as lipoid nephrosis or nil disease. In some embodiments, a method of treating or reducing the risk of minimal change disease comprises administering to a patient in need thereof an effective amount of a compound disclosed herein. Generally, minimal change disease is a kidney disease arising from a histopathologic lesion in the glomerulus and is characterized by proteinuria leading to edema and intravascular volume depletion. Minimal change disease is a common form of nephrotic syndrome.

In some embodiments, the minimal change disease treated is associated with nephrotic syndrome.

In some embodiments, the minimal change disease treated is concurrent with proteinuria, particularly excessive proteinuria.

Minimal change disease can also advance to focal segmental glomerulosclerosis.

Accordingly, in some embodiments, a method of the disclosure is directed to treatment of focal segmental glomerulosclerosis (FGS). In some embodiments, a method of treating or reducing the risk of FGS comprises administering to a patient in need thereof an effective amount of a compound disclosed herein. Generally, FGS describes both a common lesion in progressive kidney disease and excessive proteinuria and podocyte injury. The injury and scarring of the kidney is characterized by focal involvement in a segmental pattern. FGS is also a common cause of nephrotic syndrome.

In some embodiments, the FSGS treated is primary FSGS.

In some embodiments, the FSGS treated is secondary FSGS.

In some embodiments, the FSGS treated is familial FSGS. Autosomal dominant FSGS is associated with mutations in the gene encoding Inverted Formin 2 (INF2), alpha-actinin-4 gene ACTN4; the gene encoding TRPC6 cation channel protein; and the gene ARHGAP24 encoding the FilGAP protein (see, e.g., Pollak, M. R., Adv Chronic Kidney Dis., 2014, 21(5): 422-425). Recessive forms of FSGS are associated with mutations in the gene NPHS1 encoding nephrin; and the gene PLCE1 encoding phospholipase C epsilon 1 (see, e.g., Pollak, supra).

In some embodiments, the FSGS treated is associated with nephrotic syndrome.

In some embodiments, the FSGS treated is concurrent with kidney failure and/or proteinuria, particularly excessive proteinuria.

In some embodiments, the patient treated for FSGS has a prior history of minimal change disease.

As further discussed below, the compound or pharmaceutically acceptable salt thereof described herein can be administered systemically to treat the indications described herein. In some embodiments, the compound or pharmaceutically acceptable salt thereof is administered orally.

In some embodiments, the compound is I-1 or a pharmaceutically acceptable salt thereof. In some embodiments, the compound is I-2 or a pharmaceutically acceptable salt thereof.

### 4. Pharmaceutical Compositions, Administration, and Dosages

The compounds and compositions, according to the methods of the present invention, are administered using any amount and any route of administration effective for treating or lessening the severity of a disease provided above. The exact amount required will vary from subject to subject, depending on the species, age, and general condition of the subject, the severity of the infection, the particular agent, its mode of administration, and the like. Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the disease being treated. In certain embodiments, the compounds of the invention are administered orally or parenterally at dosage levels of about 0.01 mg/kg to about 50 mg/kg and for example from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present invention is directed to a composition, as described herein, comprising a prodrug of a disclosed compound. The term “prodrug,” as used herein, means a compound that is convertible in vivo by metabolic means (e.g. by hydrolysis) to a compound. Various general forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, croscarmellose or its sodium salt, and the like. Diluents, include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A therapeutically effective dose, of a compound described herein in an oral formulation, may vary from 0.01 mg/kg to 50 mg/kg patient body weight per day, more particularly 0.01 to 10 mg/kg, which can be administered in single or multiple doses per day. For oral administration, the drug can be delivered in the form of tablets or capsules containing 1 mg to 500 mg of the active ingredient specifically, 1 mg, 5 mg, 10 mg, 20 mg, 50 mg, 100 mg, 250 mg, and 500 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

For administration by inhalation, the compounds can be delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Parenteral formulations comprising a compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as by injection, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

In some embodiments, the compound or pharmaceutically acceptable salt thereof is administered systemically. In some embodiments, the compound or pharmaceutically acceptable salt thereof is administered orally.

In some embodiments, the dose of the compound or pharmaceutically acceptable salt thereof is about 10 mg to about 10,000 mg per day. In some embodiments, the dose of the compound or pharmaceutically acceptable salt thereof is about 10 mg to about 7500 mg per day. In some embodiments, the dose of the compound or pharmaceutically acceptable salt thereof is about 50 mg to about 3600 mg per day. In some embodiments, the dose of the compound or pharmaceutically acceptable salt thereof is about 250 mg to about 2400 mg per day. In some embodiments, the dose of the compound or pharmaceutically acceptable salt thereof is about 600 mg to about 5000 mg per day. In some embodiments, the dose of the compound or pharmaceutically acceptable salt thereof is about 1000 mg to about 7500 mg per day.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is about 600 mg BID (i.e., twice per day); 1.2 g BID; or 2.4 g BID.

All publications, patents, patent applications and other documents cited in this application are hereby incorporated by reference in their entireties for all purposes to the same extent as if each individual publication, patent, patent application or other document were individually indicated to be incorporated by reference for all purposes

All features of each of the aspects of the invention apply to all other aspects mutatis mutandis.

### EXEMPLIFICATION

### Example 1: A Double Masked, Placebo Controlled, Single Center, Randomized Clinical Trial to Assess the Safety and Efficacy of Compound I-1 in Subjects with Mild Asthma Induced by the Bronchial Allergen Challenge (BAC)

Study Summary

Study Phase: Phase 2

Study Objectives: Primary objective: To assess the safety of compound I-1 in subjects with allergen-induced mild asthma. Secondary objective: To assess the clinical efficacy of I-1 in subjects with allergen-induced mild asthma.

Study Endpoint:

Safety Endpoint:
Safety, as assessed by adverse events (AEs) and serious adverse events (SAEs)

Efficacy Endpoints:
Change from baseline (within visit) in forced expiratory volume in one second (FEV₁) to post-BAC (Bronchial Allergen Challenge) (during 0-3 h post-BAC [Key Efficacy Endpoint] and 3-7 h post BAC).
Absolute count and percentage differential count of sputum eosinophils and neutrophils at approximately 7 h and 24 h post-BAC.
Allergen-induced shift in airway hyper responsiveness (AHR) as assessed by Methacholine PC₂₀ (Mch PC₂₀) post-BAC.
Change from baseline in fractional exhaled Nitric Oxide (FeNO) at approximately 7 h and 24 h post-BAC.

Exploratory Endpoints:
Biomarkers (Reactive Aldehyde Species [RASP] and endotoxin-induced cytokine release) pre-BAC (at approximately 1 hour post-dose) and 7 h post-BAC.
Area under curve (AUC) of FEV1 during 0-3 h post-BAC and/or 3-7 h post BAC.

Study Population: Adult subjects with cat or house dust mite (HDM) allergen-induced mild asthma.

Study Design: A double-masked, cross-over, placebo-controlled, single center, randomized clinical trial to assess the clinical safety and efficacy of compound I-1 compared to placebo in mild cat or HDM-induced asthmatics using the BAC model. The clinical trial will consist of 9 visits to the clinic (Visits 1, 2a, 2b, 2c, 3, 4a, 4b, 5a, and 5b) over a period of approximately 75 days. During this period there will be 4 additional visits, 1 visit for safety lab and 3 visits for COVID-19 testing, as described below.

Study Products and Treatment Arms:

Treatment A: Compound I-1, 600 mg (2×300 mg tablets), orally twice daily (PO bid) for minimum of 1 week (+3).

Treatment B: Placebo, 600 mg (2×300 mg tablets), orally twice daily (PO bid) for minimum 1 week (+3).

During Post-Treatment Period 1 and 2 (Visits 4a, 4b, 5a, and 5b), in lieu of the morning dose, 600 mg of the treatments will be administered approximately one hour prior to MCT or BAC testing.

Route of Administration: Oral

Study Population: Enough subjects will be enrolled to ensure approximately 12 subjects complete the study. Subjects will be randomized (1:1) to one of the following sequences:

### 1. AB (N=6)

### 2. BA (N=6)

Study Conduct:

I. Medical Screening—Visit 1

All subjects will undergo a screening visit (Visit 1), which will include a written informed consent, demographics, medical/surgical/social/medication histories, vital signs, samples for standard clinical labs, Electrocardiogram (ECG), and a physical examination with height, weight and BMI. An asthma control questionnaire will be completed. A urine pregnancy test will be administered to women of childbearing potential (WOCBP). A skin prick test (SPT) will be performed to show positivity to cat or HDM allergen (≥3 mm wheal compared to negative control). Subjects will undergo spirometry to demonstrate baseline (pre-bronchodilator) FEV₁ of ≥80% of the predicted value. All lung function tests will be conducted in accordance with the site standard procedures (which is based on the American Thoracic Society/European Thoracic Society [ATS/ERS] recommendations). Post-bronchodilator FEV₁ will be measured within 15±5 minutes following 400 μg (4 puffs) of salbutamol inhalation and post-bronchodilator reversibility will be recorded.

II. Pre-Treatment Period (for 3 Consecutive Days)—Visits 2a, 2b and 2c

Subjects will return to the clinic for the pre-treatment period within approximately 4 weeks of the Screening visit. In all visits, staff will update the subjects' concomitant medication and collect adverse events and vital signs. Eligibility criteria will be reviewed.

At Visit 2a, asthma control questionnaire will be completed. Spirometry will be performed to ensure FEV₁≥80% of the predicted value. Subjects will have a pre-BAC Methacholine challenge test (MCT) performed as per the site standard procedures. Subjects will inhale normal saline and have a baseline FEV₁ established. Subjects will then be given subsequent doubling concentrations of Methacholine (Mch) as per the site standard procedures; 0.03 mg/mL, 0.06 mg/mL, 0.125 mg/mL, 0.25 mg/mL, 0.50 mg/mL, 1 mg/mL, 2 mg/mL, 4 mg/mL. FEV₁ will be measured at approximately 30 and 90 seconds following nebulization. If the FEV₁ drops <20%, the subject will be given the next highest concentration and spirometry repeated. Mch doses will continue to be administered sequentially (max concentration 4 mg/mL) until FEV₁ falls ≥20% of the baseline. At such time, the test will be terminated and subjects will be given 4 puffs of salbutamol, followed by a 15±5 minute waiting period prior to FEV₁ measurement. Subjects whose FEV₁ levels are not within 10% of their baseline will be given another dose of salbutamol and spirometry measurement repeated after 15±5 minutes. Mch PC₂₀ will then be calculated. All MCT will be performed at the same time of the day within a timeframe of 1.5 hours throughout the entire study. Those who qualify will undergo a multi-skin prick sensitivity test with doubling concentrations of cat/HDM allergen extracts. A positive control and a negative control will also be administered. The wheal diameters will be measured as per the site standard procedures.

At Visit 2a, those subjects who complete MCT and continue to be eligible to participate in the study will undergo a multi-skin prick sensitivity test. This test will employ similar procedures as SPT. However, multi-skin prick sensitivity test will be done using doubling concentrations of cat/HDM allergen extracts as shown in table below.

Preparation of serial dilutions using a single allergen solution

| | | Cat/HDM allergen | | |
|---|---|---|---|---|
| | Normal Saline | extracts | | |
| Volume | Added | Concentration | | Solution |
| 3 mL of 10,000 BAU/mL | 3.0 mL | 5000 | BAU/mL | A |
| 3 mL of dilution A | 3.0 mL | 2500 | BAU/mL | B |
| 3 mL of dilution B | 3.0 mL | 1250 | BAU/mL | C |
| 3 mL of dilution C | 3.0 mL | 625 | BAU/mL | D |
| 3 mL of dilution D | 3.0 mL | 312.5 | BAU/mL | E |
| 3 mL of dilution E | 3.0 mL | 156.25 | BAU/mL | F |
| 3 mL of dilution F | 3.0 mL | 78.13 | BAU/mL | G |
| 3 mL of dilution G | 3.0 mL | 39.06 | BAU/mL | H |
| 3 mL of dilution H | 3.0 mL | 19.53 | BAU/mL | I |
| 3 mL of dilution I | 3.0 mL | 9.77 | BAU/mL | J |

The wheal diameter will be measured as per the site standard procedures. Skin sensitivity will be defined as the lowest allergen concentration that produces a wheal of ≥3 mm in diameter relative to the negative control.

Salbutamol inhaler with spacer (rescue medication) will be provided to the subject to take home at Visit 2a and rechecked at every Clinic Visit to ensure that the subjects have enough medication. The subjects will be issued a diary (including Asthma Action Plan) to keep a daily log of any changes in their health or medication use (including rescue medication) in their diary while at home. The subjects will be given the option to be confined at the study site in order to facilitate early morning visit on the next day.

The next day (Visit 2b), subjects will undergo a BAC. At Visit 2b, the subjects' old diary (including Asthma Action Plan) will be collected and the subjects will be issued a new diary to keep a daily log of any changes in their health or medication use (including rescue medication) while at home. The allergen concentration to be administered will be determined based on the results from the MCT and allergy SPT titrations performed at Visit 2a.

The concentration of the allergen provocative concentration (PC₂₀) will then be predicted from the previous Mch PC₂₀ and the skin sensitivity using the following logarithmic formula: Log 10 (Allergen PC₂₀)=0.68 log 10 (Mch PC₂₀×skin sensitivity) where skin sensitivity is the skin prick test end point dilution titration which is the lowest concentration producing a ≥3 mm wheal. (This formula may be subject to modification and is dependent on the allergen PC₂₀ estimated in subjects).

Following the calculation of the predicated allergen PC₂₀, subjects will first inhale diluent saline for a period of 1 minute by tidal breathing and FEV₁ will be measured at approximately 30 and 90 seconds post inhalation. The higher of the 2 FEV₁ measurements with the saline diluent will be used as the baseline value. Subjects will then be given 3 consecutive doubling doses of allergen below that predicted to induce a 20% fall in PC₂₀ for safety. Subjects will be administered the allergen as per the site standard procedures. At approximately 10 minutes post inhalation of the first allergen dose, a duplicate FEV₁ will be measured. If the FEV₁ has dropped <10% from baseline, the next allergen concentration can be delivered and subsequent doubling step up doses (each 2-fold greater than previous concentration) until 20% FEV₁ reduction from baseline is reached. A duplicate FEV₁ will be measured at approximately 10 minutes post inhalation of each allergen dose. During the allergen titration, if the FEV₁ has fallen between 10% and 20% from pre-allergen baseline, the FEV₁ is repeated 20 minutes after inhalation. If the FEV₁ does not fall ≥20% even after repeat FEV₁, then next dose of the allergen will be given. When a drop in FEV₁ of 20% from baseline has finally been achieved, the challenge titration will be terminated and the target allergen titer will be recorded.

Early phase asthmatic response (EAR) is defined as a ≥20% fall in FEV₁ from the highest pre-inhalation FEV₁ value on at least one occasion within 3 h after the inhalation of the final concentration of allergen. In order to assess the EAR, FEV₁ will be measured at approximately 30, 60, 90, 120, and 180 minutes post allergen exposure. Late phase asthmatic response (LAR) is defined as a ≥15% fall in FEV₁ from the highest pre-inhalation FEV₁ value on at least one occasion between 3 and 7 h after the inhalation of the final concentration of allergen. To assess the LAR, FEV₁ will be measured every hour between 3 to 7 hours post allergen challenge.

At the end of the monitoring period, 4 puffs of bronchodilator (salbutamol) will be administered to the subjects to restore FEV₁ to 90% of pretest FEV₁, if necessary. If FEV₁ does not return to normal levels, the Investigator/medical designee will assess the subject. Following the test, sputum will be induced, collected, and processed (approximately 7 h post-BAC). The subjects will be given the option to be confined at the study site in order to facilitate early morning visit on the next day.

The next day (Visit 2c) sputum induction and collection (approximately 24 h post-BAC) will be performed. Additionally, a blood sample will be taken and sent to analytical lab for exploratory biomarkers (RASP and endotoxin-induced cytokine release). This will be considered as baseline value.

At Visit 2c, the subjects' old diary will be collected and the subjects will be issued a new diary to keep a daily log of any changes in their health or medication use (including rescue medication) while at home. Subjects will also receive an asthma action plan to monitor asthma symptoms. Subjects will be asked to return to the clinic after approximately 2 weeks.

Note: Safety clinical laboratory tests will be repeated within 3 days prior to the first dose of study drug to ensure continued eligibility.

III. Randomization Visit—Visit 3

Following at least 2 weeks wash-out period, eligible subjects will return to the clinic for Visit 3 to participate in the treatment periods. Clinic staff will update the subjects' concomitant medication and collect AEs and vital signs. Eligibility criteria will be reviewed.

Diary cards including asthma action plan will be collected and reviewed and subjects will be issued anew diary. Asthma control questionnaire will be collected. A urine pregnancy test will be administered to WOCBP.

Subjects will be randomized to either Sequence treatment AB or Sequence Treatment BA. Subjects will be dispensed compound I-1 or Placebo for at-home treatment with instructions for dosing. Subjects will receive their first dose on site.

Blood sample will be collected for PK assessment at 1 hour (+5 minutes) post dose.

An Electrocardiogram (ECG) will be performed at 1 hour (+15 minutes) post dose.

IV. Treatment Period 1

At home, subjects will take the treatment (Treatment A or Treatment B) orally twice per day, i.e. PO bid dosing for minimum 1 week (+3 days) and return to the Clinic for the Post-Treatment Period 1. Subjects will take the morning and evening dose at approximately the same time each day. Additionally, there will be a phone call during the treatment period to follow up on subject's health and treatment compliance.

Subjects will continue to keep a daily log of any changes in their health or medication use (including rescue medication) and time of dosing in their diary while at home. They will also continue to refer to the asthma action plan, if there is any worsening of asthma control.

Additionally, subjects will receive a phone call on the last day of the treatment period to remind them that their morning dose (600 mg) of the treatment will be administered onsite next day.

V. Post-Treatment Period 1 (for 2 Consecutive Days)—Visits 4a & 4b

Subjects will not stop treatment in order to maintain steady state concentration of the drug during the Visits 4a and 4b. Hence subjects will continue to receive their respective treatments with same schedule. However, on the days of visits 4a and 4b, subjects will receive their morning dose (600 mg) of the treatment on site approximately one hour prior to MCT or BAC.

Staff will update the subjects' concomitant medication and collect AEs and vital signs. Asthma control questionnaire will be collected. Eligibility criteria will be reviewed. Diary cards including asthma action plan will be collected and reviewed and subjects will be issued a new diary. Blood and urine samples will be collected for safety clinical laboratory tests (CBC with differential, electrolytes [Calcium, Sodium, Potassium, Chloride], eGFR, creatinine, BUN, ALT, AST, ALP, total bilirubin, albumin, total protein, glucose, total cholesterol, triglycerides, lipase and amylase and urinalysis including assessment of microalbuminuria).

At Visit 4a, pre-BAC FeNO (baseline) and baseline FEV₁ will be performed. Pre-BAC FeNO to be performed prior to baseline FEV₁. At approximately 1 hour post-dose blood samples will be taken and sent to analytical lab for exploratory biomarkers (RASP and endotoxin-induced cytokine release) prior to BAC. An additional blood sample will be collected for PK assessment at 1 hour (+5 minutes) post dose. An ECG will be performed at 1 hour (+15 minutes) post dose.

Subjects will then undergo BAC with the target allergen titer dilution identified in the pre-treatment allergen challenge visit (Visit 2b). Approximately 7 h post-BAC, FeNO will be measured and then sputum will be induced, collected, and processed. FeNO to be performed prior to 7 h FEV₁ measurements. Additionally, blood samples will be taken and sent to analytical lab for exploratory biomarkers (RASP and endotoxin-induced cytokine release), at approximately 7 h post-BAC. The subjects will be given the option to be confined at the study site in order to facilitate early morning visit on the next day. At home treatment will be dispensed and/or collected based on subject's decision regarding confinement.

Once all procedures are completed, subjects will be reminded to take their next day morning dose on site approximately one hour prior to MCT.

The following day (Visit 4b), subjects will undergo post-BAC MCT, FeNO and sputum testing (approximately 24 h post-BAC). FeNO will be performed before any other procedure on Visit 4b. Procedures for MCT (including baseline FEV₁) will be repeated as described in Visit 2a, except that the maximum concentration of Mch used in this visit will be up to 16 mg/mL. Following MCT, sputum will be induced, collected, and processed (approximately 24 h post-BAC). Any remaining at home treatment will be collected.

After completion of Visit 4b study procedures, subjects will be dispensed the second treatment according to their assigned sequence and new at-home diary cards and will be asked to follow the same instructions as previously directed. Following Visit 4b, subjects will complete 2 weeks of washout period. Subjects will continue to keep a daily log any of any changes in their health or medication use (including rescue medication) and time of dosing in their diary while at home. They will also continue to refer to the asthma action plan, if there is any worsening of asthma control.

Subjects will receive a phone call approximately 1 day prior to their scheduled initiation of at-home dosing as a reminder to start treatment. Staff will update the subjects' concomitant medication and rescue medication use and collect AEs.

VII. Treatment Period 2

At home, subjects will take the treatment (Treatment B or Treatment A) orally twice per day, i.e. PO bid dosing for minimum 1 week (+3 days) and return to the Clinic for the Post-Treatment Period 2. Subjects will take the morning and evening dose at the same time each day. Additionally, there will be a phone call during the treatment period to follow up on subject's health and treatment compliance.

Subjects will continue to keep a daily log of any changes in their health or medication use (including rescue medication) and time of dosing in their new diary while at home. They will also continue to refer to the asthma action plan, if there is any worsening of asthma control.

Additionally, subjects will receive a phone call on the last day of the treatment period to remind them that their morning dose (600 mg) of the treatment will be administered onsite next day.

VIII. Post-Treatment Period 2 (For 2 consecutive days)—Visits 5a & 5b

Subjects will return to the Clinic for the Post-Treatment Period 2 following approximately 3 weeks (+3 days) later after having completed 2 weeks of washout and 1 week (+3 days) of at-home dosing. At Visits 5a and 5b, subjects will follow same procedures as performed previously at Visits 4a and 4b, respectively. As with Visits 4a and 4b, on the days of visits 5a and 5b, subjects will receive their morning dose (600 mg) of the treatment on site approximately one hour prior to MCT or BAC.

Additionally, at Visit 5b, paper diary cards including asthma action plan and any remaining at home treatment will be collected from the subjects and a urine pregnancy test will be administered to WOCBP.

Subjects will complete a health check prior to clinical trial exit.

IX. Early Termination Visit (ETV)

Staff will update the subjects' concomitant medication and collect any unused at home treatments, adverse events, asthma control questionnaire, and vital signs. Paper diaries will be collected and reviewed. Urine pregnancy test for WOCBP only will be done, if not performed before on the same day.

Inclusion Criteria:

1. Male or non-pregnant female, between 18 to 65 years of age (inclusive) at Screening Visit.
2. Subjects must give their signed and dated written informed consent (in English) to participate prior to commencing any study-related activities and must be willing to comply with study procedures, study restrictions, study protocol, and return for the required assessments.
3. Female subjects of either non-childbearing potential or of child-bearing potential who commit to consistent and correct use of at least one highly effective or two effective forms of contraception starting at least 4 weeks prior to the Screening Visit and for at least 30 days post last dose of study drug.
4. Generally healthy subjects with mild controlled asthma for 2 years at Screening Visit according to the Global Initiative for Asthma (GINA 2020) criteria.
5. No concomitant asthma treatment, except inhaled SABA.
6. Positive SPT to HDM (Dermatophagoides pteronyssinus and/or Dermatophagoides fariniae) or cat (Felis domesticus) allergen (≥3 mm wheal compared to negative control).
7. Baseline FEV₁≥80% of the predicted normal after withholding SABA for >6 hours.
8. Demonstrate a ≥20% decrease in FEV₁ in the pre-treatment MCT at a dose of ≤4 mg/mL at Clinic Visit 2a.
9. Currently a non-smoker; having not used tobacco products (i.e. cigarettes, cigars, pipe tobacco) within the past year, and having ≤10 pack-years of historical use. Use of electronic cigarettes or other inhaled nicotine delivery products, smoking and/or inhalation of cannabis using a device (e.g. vaping) will not be allowed during the study.
10. Agree to limit caffeine and consumption of cruciferous vegetables and grilled meats. Agree to prohibit concomitant medications (strong CYP1A2, 2B6 and 3A4 inhibitors).
11. Body mass index (BMI) within the range 18.5-35.0 kg/m².
12. Male subjects who commit to not father a child or donate sperm from first dose until 3 months post-last dose.
13. Male subjects (with female partners of childbearing potential) who commit to consistent and correct use of at least two effective methods of birth control for the duration of the study and 30 days after the last dose of study drug.
14. AST, ALT, ALP, TSH, White Blood Count, hemoglobin, glucose, albumin, electrolytes, total proteins and total bilirubin within the normal range.
15. Acceptable lipase, amylase, GGT, CPK, total cholesterol, triglycerides, and eosinophils levels as determined by the Investigator in consultation with the medical monitor.
16. Normal renal function with eGFR >90 ml/min/1.73 m².
17. Heart rate within 50-90 bpm. (Note: In order to include subjects with bpm <50 and ≥45 bpm they should have normal thyroid function [medical history, physical examination, TSH] and no signs of diseases associated with bradycardia [e.g., orthostasis and dizziness]).

Exclusion Criteria:

1. History and presence of clinically significant cardiovascular, renal, neurologic, hepatologic, endocrinologic, gastrointestinal, genitourinary, autoimmune, hematological, or metabolic disease other than asthma, which in the opinion of Investigator may either put the subject at risk or influence the results during the study.
2. Subjects with perennial allergy symptoms and/or possible exposure to perennial allergens (e.g. mold, dog) that occur or are anticipated to occur during the study at the discretion of the investigator. Subjects with seasonal allergy symptoms that occur or are anticipated to occur during the study should result in subject exclusion or rescheduling until the subject is out of the allergy season.
3. Any relevant pulmonary disease within 1 year prior to dosing at the discretion of the investigator.
4. Recent hospitalization with asthma in the last 6 months or any other medical condition that the Investigator deems incompatible with participation in the trial.
5. Inability to tolerate temporary withdrawal of current asthma medication.
6. Other co-morbid respiratory and sinus diseases.
7. History of frequent asthma exacerbations in the previous year.
8. The use of the following medications: beta blockers, tricyclic/polycyclic antidepressants, monoamine oxidase inhibitors within 14 days of the study.
9. History or current evidence of clinically relevant allergies or idiosyncrasy to drugs.
10. Known intolerance or hypersensitivity to any component of the salbutamol MDI and intolerance to aerosolized β₂-adrenergic agonists.
11. Female subjects of child-bearing potential who are pregnant, lactating or using inadequate contraceptive measures.
12. Subjects that have a history of alcohol, drug or medication abuse within the past year before the study.
13. Subjects that lack cooperation or compliance, as judged by the Investigator.
14. Subjects suffering from severe psychiatric, psychological, or neurological disorders.
15. Subjects who are employees of the sponsor or CRO and/or 1st grade relatives or partners of the (principal) Investigator.
16. Inability to demonstrate the proper use of the nebulizer as determined by the staff.
17. Any clinically significant abnormal finding on the physical examination, vital signs or laboratory results at screening as deemed so by the Investigator.
18. The use of any investigational drug within 30 days of the study.
19. Allergen immunotherapy treatment with cat or HDM within the previous 5 years.
20. Any clinically significant physical findings of nasal anatomical deformities (including the presence of nasal mucosal ulceration, nasal polyps, purulent secretions, septal perforation or any other major abnormalities in the nose), which at the discretion of the Investigator, would interfere with the study procedures.
21. Any surgery requiring general anaesthesia three months before the Screening Visit or planned during the study period.
22. Known hypersensitivity to compound I-1 or any of its formulation components.
23. History of anaphylaxis or angioedema.
24. Previous history of life-threatening asthma and/or exacerbation of asthma within 6 weeks prior to the Screening Visit.
25. Previous history of respiratory tract infection within 2 weeks prior to the Screening Visit.
26. History of risk factors for TdP (e.g., heart failure, hypokalemia, family history of long QT syndrome).
27. Persistent systolic BP >140 mmHg or diastolic BP >90 mmHg.

### 28. At Screening Visit, QTcF >450 ms.

29. Public health emergency (e.g., COVID-19): subject not complying with Public health guidelines (e.g., self-isolation), at the discretion of the Investigator and/or designee, or subjects with a positive COVID-19 test result up to 5 days prior to Visit 2a, Visit 4a or Visit 5a.

Statistical Analysis:

The safety endpoint will be summarized descriptively.

The key efficacy endpoint change from baseline in FEV₁ during 0-3 h post-BAC may be analyzed using a mixed effect model for repeated measures (MMRM) with the following independent factors, within-visit baseline FEV₁ as covariate, and sequence, visit, treatment, post-BAC assessment time, and interaction of treatment by post-BAC assessment time. Subject may be treated as a random effect. If deemed appropriate, baseline sputum eosinophil count may be included as an additional covariate in MMRM.

AUCs may be analyzed using a mixed effect model with following terms: sequence, visit (i.e., period), treatment group subject as random effect.

The other efficacy endpoints may be compared between treatments using appropriate statistical models.

The Statistical Analysis Plan will detail all statistical procedures and will take precedence over any statistical descriptions herein.

Safety Analysis:

All study subjects who receive at least one dose of any of the study products will be included in the comparative safety analysis. Adverse events will be classified using standard Medical Dictionary for Regulatory Activities (MedDRA) terminology Version 22 or higher and presented by treatment group. Summary tables listing the type, date of onset, date and time of resolution, incidence, severity, outcome, action taken, and Investigator's opinion of relationship to the study product will be presented by treatment group for AEs reported after randomization.

Concomitant medication used during the study will be tabulated by treatment by subject.

Sample Size Determination:

Based on repeatability analyses in allergen-induced airway inflammation responses, a sample size of 12 subjects yields more than 80% power to detect a difference of 0.1 with standard deviation of 0.1 in change from baseline FEV₁ across treatment groups.

Introduction

Background and Study Rationale

Type I allergy is an immune-disorder which results from the inappropriate formation of Immunoglobulin E (IgE) antibodies against proteins and glycoproteins from plants, insects, animals, and fungi, most of which are normally considered harmless. The cross-linking of IgE antibodies on effector B cells by allergens activates an immunological cascade leading to some or all of the symptoms of Type I allergy which may include rhinitis, conjunctivitis, asthma, and anaphylactic shock.

Asthma is a serious global health problem and one of the most common diseases in the Western world. Allergic asthma is the most common form of asthma, with over 50% of the asthma population being affected by allergic asthma. Asthma is a chronic inflammatory disorder of the airways in which a variety of cell types and cellular elements play a role. Airway inflammation produces four forms of airflow limitations: acute bronchoconstriction, swelling of the airway wall, mucus hypersecretion, and airway wall remodeling. The chronic inflammation causes an associated increase in airway hyper-responsiveness that leads to recurrent episodes of wheezing, breathlessness, chest tightness, and coughing. These episodes are usually associated with widespread, but variable airflow obstruction that is often reversible, either spontaneously or with treatment.

Bronchial allergen challenge (BAC) testing is known as the “gold standard” for the investigation of allergic asthma and has been used for almost 3 decades. Safely and properly performed BAC model offers a valuable tool for assessing a drug's clinical efficacy in a small sample size of subjects.

This validated model mimics the acute and some of the more chronic features of asthma as well as aids in the understanding of the blocking effects of investigational therapies. The classical approach that has been routinely used is by which subjects with allergic rhinitis are challenged with the same amount of allergen before and after treatment with a specific agent.

Common aeroallergens such as house dust mite, pollen, mold, and animal dander are not only well-known contributors to airway inflammation in allergic asthma but are known causal agents of persistent asthma and exacerbations of asthma. Re-exposure to any of these triggers occurs due to the binding and cross-linking of allergen to IgE bound to the mast cells and basophils. The subsequent degranulation of these cells can lead to the immediate release of Type I hypersensitivity mediators including histamine, leukotrienes and prostaglandins. In turn, this inflammatory cascade induces the direct contraction of acute airflow obstruction, and asthmatic symptoms associated with wheezing, coughing and dyspnea. This stage is known as the early phase asthmatic response (EAR) and within 15 to 30 minutes of the exposure and usually resolves by about 2-3 hours. Episodes of recurrent bronchoconstriction occurring between 3 and 12 hours involving the further activation of a variety of recruited inflammatory cells and monocytes and the production of cytokines is carried on into what is known as the late phase asthmatic response (LAR). As the disease becomes more persistent, the inflammatory profile changes and becomes more progressive, with a greater involvement of neutrophils, edema, and mucus hypersecretion and increase airway hyperresponsiveness.

Here we present a study utilizing the BAC model to induce asthma. In this design, during post-treatment periods, Methacholine challenge Test (MCT) will be performed post allergen challenge (separated by a 24 hour time period) which provides another useful outcome for the evaluation of the allergen-induced airway hyperresponsiveness. Also sputum will be induced after each MCT which will allow for the determination of eosinophils and neutrophils. Allergen-induced sputum eosinophilia is a useful measurement in the assessment of the anti-inflammatory properties of asthma therapies.

Compounds such as I-1 are small molecules with a quinoline core that act as a reactive aldehyde species (RASP) inhibitor by irreversibly binding to RASP. Compound I-1 and others in its class are useful in the treatment of systemic immune-mediated and inflammatory diseases, including psoriasis, inflammatory bowel disease, asthma, ulcerative colitis, non-alcoholic steatohepatitis, and other diseases believed to be caused, or exacerbated, by elevated concentrations of RASP.

Free RASP (e.g., malondialdehyde [MDA] and 4-hydroxynonenal [FINE]) are toxic, leading to inflammation and molecular dysfunction by reacting with cellular biomolecules, and have been implicated in many immune-mediated and inflammatory diseases. Quinoline compounds such as I-1 bind to free RASP via a rapid, two-step reaction involving Schiff base formation followed by a ring closure, resulting in stable and non-reactive adducts that are subsequently degraded.

The potential benefit of RASP inhibition in immune-mediated and inflammatory diseases has been demonstrated by the first-in-class RASP inhibitor reproxalap (ADX-102), which has been shown beneficial in treating ocular inflammation, including dry eye disease and allergic conjunctivitis across numerous Phase 2 and Phase 3 clinical trials, and is now in Phase 3 clinical testing.

Secondary pharmacology studies, which include a large panel of ligand binding assays, ion channel assays, transporter assays, and enzyme inhibition studies, suggest that there is a low risk of off-target effects due to treatment with compound I-1. In addition, in vitro studies have shown that I-1 has a very low potential to inhibit the delayed rectifier potassium current. Results of preclinical studies demonstrate that I-1 has a low risk of genotoxicity. I-1 plasma concentrations are projected to have reached at least 10 μM, exceeding reported levels of RASP in humans with inflammatory diseases. The data support the potential of I-1, and RASP inhibition in general, in treating inflammation and fibrosis. Genotoxicity studies have shown no potential for mutagenicity or clastogenicity of I-1.

In a first-in-human, randomized, double-blind, placebo-controlled Phase 1 trial, compound I-1 was found to be safe and tolerable. The adverse event profile was favorable compared to placebo: A total of 6 (9.4%) subjects receiving the test compound had treatment emergent adverse events, compared to 4 (19.1%) subjects who received placebo. There were no interruptions or discontinuations of study drug administration. No clinically meaningful changes were observed in hepatic or renal analytes, including transaminases (ALT and AST), alkaline phosphatase (ALP), amylase, gamma-glutamyl transpeptidase (GGT), bilirubin, creatinine kinase and creatinine. No changes in serum glucose were observed. No clinically meaningful changes were observed in heart rate (HR), blood pressure (systolic, diastolic and orthostatic changes), respiratory rate, pulse oximetry, or temperature. No clinically significant hematological changes were observed. The compound did not lead to QTcF prolongation. There were no subjects who had QTcF >500 msec or a change of >60 msec from baseline. Five subjects had a change of >30 msec from baseline but did not require intervention or study drug interruption or discontinuation, and all subjects remained asymptomatic. Three of these five subjects were in the SAD portion of the study (one each in the 100 mg, 200 mg, and 700 mg dose cohorts) and the remaining two subjects were in the MAD portion of the study (one each in the 150 mg BID and 300 mg BID dose cohorts).

Healthy volunteers were dosed with 600 mg of compound I-1 BID for 10 days in the top (highest) dose cohort. The observed Cmax on Day 1 was 1920 ng/mL (67.4% CV) and 1458 ng/mL (46.6% CV) on Day 10. The 0 to 12 hour area under the curve (AUCtau) was 5710 h*ng/mL (61.5% CV) on Day 1 and 6,800 h*ng/mL (37.9% CV) on Day 10. The observed half-life of the drug was 3.98 h (22.9% CV) on Day 1 and 4.56 h (12.1% CV) on Day 10.

These results indicate that no cyp autoinduction was observed over the course of 10 days as no significant increase in clearance was observed between Day 1 and Day 10. Although pharmacokinetic (PK) variability was evident, a linear correlation was observed in Cₘₐₓ and AUC as dose increased. The half-life (t_{1/2}) was consistent across cohorts and days, and mean values in multiple day exposures ranged between 3.07 to 6.20 hours. Little to no accumulation of the drug was seen across all cohorts.

A decrease in free MDA levels was observed in the plasma of healthy volunteers over 10 days of dosing with compound I-1 600 mg BID that was statistically greater than that of subjects treated with placebo. Following ingestion of a high-fat meal on Day 10 of dosing with 600 mg BID or placebo, levels of free fatty acids were statistically lower and levels of HDL were statistically higher in drug-treated subjects than in placebo-treated subjects, potentially representing additional anti-inflammatory activity of compound I-1.

Rationale for Dose Selection

Clinical development of compound I-1 in inflammatory disease is supported by safety testing in human healthy volunteers in single and multiple ascending dose (10 day), placebo-controlled Phase I trials. Overall, the compound was found to be safe and tolerable at the doses explored, including the maximum dose of 600 mg BID.

The dose for Phase 2 clinical trials of compound I-1 of 300 mg BID PO is based on conservative margins from 28-day nonclinical assessments and drug exposure in humans in the Phase 1 clinical trial, which generally exceeds levels of RASP reported in human inflammatory disease.

Objectives and Endpoints

Primary Objective:

To assess the safety of compound I-1 in subjects with allergen-induced mild asthma.

Secondary Objective:

To assess the clinical efficacy of compound I-1 in subjects with allergen-induced mild asthma.

Safety Endpoint:

Safety, as assessed by adverse events (AEs) and serious adverse events (SAEs)

Efficacy Endpoints:
Change from baseline (within visit) in forced expiratory volume in one second (FEV1) to post-BAC (during 0-3 h post-BAC [Key Efficacy Endpoint] and 3-7 h post BAC).
Absolute count and percentage differential count of sputum eosinophils and neutrophils at approximately 7 h and 24 h post-BAC.
Allergen-induced shift in airway hyper responsiveness (AHR) as assessed by Methacholine PC20 (Mch PC20) post-BAC.
Change from baseline in fractional exhaled Nitric Oxide (FeNO) at approximately 7 h and 24 h post-BAC.

Exploratory Endpoints:
Biomarkers (RASP and endotoxin-induced cytokine release) pre-BAC (at approximately 1 hour post-dose) and 7 h post-BAC.
Area under curve (AUC) of FEV1 during 0-3 h post-BAC and/or 3-7 h post BAC.

Clinical Trial Design

This trial is double-masked, cross-over, placebo-controlled, single center, randomized clinical trial to assess the clinical safety and efficacy of compound I-1 compared to placebo in mild cat or HDM-induced asthmatics using the BAC model. The study will consist of 9 visits to the clinic (Visits 1, 2a, 2b, 2c, 3, 4a, 4b, 5a, and 5b) over a period of approximately 75 days. During this period there will be 4 additional visits, 1 visit for safety lab and 3 visits for COVID-19 testing, as described below. The clinical trial will be conducted as follows:
1. Medical Screening: Visit 1
2. COVID-19 test within 5 days prior to Pre-Treatment Period
3. Pre-Treatment Period (For 3 consecutive days)
   a. Visit 2a
   b. Visit 2b
   c. Visit 2c
4. Washout (2 weeks)
5. Additional visit for safety sample blood collection within 3 days of Visit 3
6. Randomization Visit: Visit 3
7. Treatment Period 1 (at home treatment taken for 1 week [+3 days])
8. COVID-19 test prior to Post-Treatment Period 1
9. Post-Treatment Period 1 (For 2 consecutive days):
   a. Visit 4a
   b. Visit 4b
10. Washout (2 weeks)
11. Treatment Period 2 (at home treatment taken for 1 week [+3 days])
12. COVID-19 test prior to Post-Treatment Period 2
13. Post-Treatment Period 2 (For 2 consecutive days):
   a. Visit 5a
   b. Visit 5b

The end of study is defined as the time at which the last subject has completed all study procedures in the clinical trial.

Subject Selection information, including Inclusion and Exclusion Criteria, is provided above.

Study Product and Randomization

The following products will be used in the study:

Test Product (Treatment A)

2×300 mg compound I-1 tablet taken PO bid for minimum 1 week (+3 days)

Placebo (Treatment B)

2×300 mg Placebo tablet taken PO bid for minimum 1 week (+3 days)

Dosing Instructions:
The subjects should swallow the tablets with water, tablets should not be chewed.
The subjects should take each dose at least 60 mins before food.
There should be a gap of at least 4 hours between the two doses per day.
The subjects should not take broken tablets; however minor tablet defects like chipping or scratching are acceptable.
In case of an overdose, the subjects should hold the next dose and inform clinic immediately.
In case of a missed dose, the subjects should take the dose when they remember only if their next scheduled dose is not within time frame of 4 hours. If the next scheduled dose is within time frame of 4 hours then subject should not take their missed dose and take the next dose at scheduled time.
A minimum of two days of BID dosing during each treatment period are required before BAC/MCT.
During Post-Treatment Periods 1 and 2 (Visits 4a, 4b, 5a, and 5b), in lieu of the morning dose, 600 mg of the treatments will be administered approximately one hour prior to MCT or BAC testing.

Once the clinical trial has begun, the subjects will be instructed to take only the study medication(s) described in the protocol. If the subject takes any other medication during the clinical trial, the Investigator will record the necessary information and may notify the Sponsor, if judged to be significant.

Items restricted prior to and during the course of this clinical trial are described in the table below:

| | | End of |
|---|---|---|
| Restricted Item | Start of Restriction | Restriction |
| SABAs | 8 hours Prior to MCT, | — |
| | BAC | |
| Long acting bronchodilators | 48 hours prior to MCT, | End of last |
| i.e. formeterol, salmeterol | BAC | study visit |
| Ipratropium bromide | 24 hours prior to MCT, | End of last |
| | BAC | study visit |
| Tiotropium | 72 hours prior to MCT, | End of last |
| | BAC | study visit |
| Theophylline | 48 hours prior to MCT, | End of last |
| | BAC | study visit |
| Leukotriene modifiers i.e., | 7 days prior to MCT, | End of last |
| Singular (montelukast) | BAC | study visit |
| Cholinesterase inhibitor | 2 weeks prior to | End of last |
| | Screening | study visit |
| Beta blockers, | 14 days before Medical | End of last |
| tricyclic/polycyclic | Screening-Visit 1 | study visit |
| antidepressants, monoamine | | |
| oxidase inhibitors | | |
| Antihistamines i.e., | 7 days prior to the | End of last |
| cetirizine, fexofenadine or | Medical Screening- | study visit |
| loratadine | Visit 1 and Visit 2a and | |
| | 12 to 24 hours prior to | |
| | MCT, BAC | |
| Cromolyn sodium | 8 hours prior to MCT, | End of last |
| | BAC | study visit |
| Nedocromil | 48 hours prior to MCT, | End of last |
| | BAC | study visit |
| Concomitant CYP1A2, 2B6 | 24 hours prior to MCT, | End of last |
| and 3A4 Substrates | BAC | study visit |
| Strong CYP1A2, 2B6 and | 24 hours prior to MCT, | End of last |
| 3A4 inhibitors | BAC | study visit |
| Acetaminophen | 72 hours before Medical | End of last |
| | Screening-Visit 1 | study visit |
| Coffee, tea, cola, caffeinated | First dose of the study | Last dose of |
| beverages, chocolate | products for each | the study |
| (No more than one serving | treatment period | products for |
| per day) | | each treatment |
| | | period |
| Allergen immunotherapy | 5 years before Medical | End of last |
| treatment with cat or HDM | Screening Visit 1 | study visit |
| Investigational drug/product | 30 days before Medical | End of last |
| | Screening Visit 1 | study visit |
| Smoking (used tobacco | 12 months before | End of last |
| products i.e., cigarettes, | Medical Screening | study visit |
| cigars, pipe tobacco, | Visit 1 | |
| electronic cigarettes or other | | |
| inhaled nicotine delivery | | |
| product) | | |
| Smoking and/or inhalation | Screening Visit 1 | End of last |
| of cannabis using a device | | study visit |
| (e.g., vaping) | | |
| Alcohol | 12 hours before Medical | End of last |
| | Screening Visit 1 | study visit |
| Cruciferous vegetables | First dose of the study | Last dose of |
| (1-2 servings per week) | products for each | the study |
| | treatment period | products for |
| | | each treatment |
| | | period |
| Grilled meats | First dose of the study | Last dose of |
| (1-2 servings per week) | products for each | the study |
| | treatment period | products for |
| | | each treatment |
| | | period |
| Green leafy vegetables | 2 hours prior to FeNO | — |
| Eating and drinking | 1 hour prior to FeNO | — |
| anything | | |
| Any surgery requiring | 3 months before Medical | End of last |
| general anaesthesia | Screening Visit 1 | study visit |
| Oral corticosteroids | 30 days prior to Medical | End of last |
| | Screening-Visit 1 | study visit |
| Intranasal corticosteroids | 2 weeks prior to Medical | End of last |
| | Screening-Visit 1 | study visit |
| Inhaled corticosteroids | 2 weeks prior to Medical | End of last |
| | Screening-Visit 1 | study visit |
| Exposure to perennial | End of Medical | End of last |
| allergens (e.g. mold, dog) | Screening-Visit 1 | study visit |

A complete listing of medications that are CYP Inhibitors and Inducers are available online at medicine.iupui.edu/clinpharm/ddis/main-table.

No other concurrent medications, other than mentioned in the above restriction table, are allowed during the trial conduct unless deemed necessary per the investigator's medical judgement.

Subjects who violate any of the above restrictions may be excluded or dropped from the clinical trial at the discretion of the Investigator. Individual exceptions to the above restrictions may be approved by the Sponsor and/or Investigator.

### Example 2: In Vitro Model of Alcoholic Liver Injury Using Precision-Cut Liver Slices (PCLS) from Rats

Alcohol abuse results in liver injury, including accumulation of RASP and an increase in inflammation. It is known to cause abnormalities in liver structure and function, including fatty liver, apoptosis, necrosis, fibrosis, and cirrhosis. In order to investigate the utility of the quinoline compounds described herein for reduction of RASP and/or treating liver and associated inflammatory disorders, we employed a known in vitro culture model that employs precision-cut liver slices (PCLS) to measure alcohol-induced liver injury. For this experiment, we used a liver sample donated from a 6 year old female with fibrosis. This experiment may also be performed on rat PCLS (procedure provided below). The model is described, for example in Klassen, L. W. et al., Biochemical Pharmacology 76 (2008), 426-436. In this model, PCLS retains excellent viability as determined by lactate dehydrogenase and adenosine triphosphate (ATP) levels over a 96-h period of incubation. The major enzymes of ethanol detoxification (alcohol dehydrogenase, aldehyde dehydrogenase, and cytochrome P4502E1), remain active and PCLS readily metabolizes ethanol and produces acetaldehyde. Within 24 h and continuing up to 96 h, the PCLS develope fatty livers and demonstrate an increase in the redox state. These PCLS secrete albumin, and albumin secretion was decreased by ethanol treatment. All of these impairments are reversed following the addition of 4-methylpyrazole, which is an inhibitor of ethanol metabolism. Therefore, this model system appears to mimic the ethanol-induced changes in the liver that have been previously reported in human and animal studies, and appears to be a useful model for the study of alcoholic liver disease.

Materials and Methods. For human PCLS studies, slices were cut as described below and incubated with conditions below for 24, 48, or 72 hours. Media were changed daily.
Control Media
25 mM Ethanol Media.
Control Media+10 μM compound I-1 (7 μl of the 5 mg/ml I-1 solution in 1.7 ml)
Ethanol Media+10 μM compound I-1

Studies on rat PCLS may be performed as follows. Following the procedure of Klassen, L. W. et al., appropriate rats such as male Wistar rats are purchased and maintained on a standard diet. All animals are allowed free access to their food and/or water up to 1 h prior to sacrifice.

Rat precision-cut liver slices are prepared as follows. Rats weighing 200-300 g are anesthetized using Isoflurane. The basic method of Olinga et al. (Olinga P. et al., J Pharmacol Toxicol Methods 1997; 38(2):59-69) is used to prepare the PCLS. Briefly, the abdominal cavity is scrubbed with betadine and entered, exposing the liver. The inferior vena cava is clipped, blood allowed to drain for 1 min, the liver excised and quickly placed into oxygenated V-7 cold preservation buffer (commercially available from Vitron Inc., Tucson, Ariz.). Multiple (8 mm) cylindrical tissue cores are cut using a hand held coring tool, loaded into a tissue slicer, and 250 mm thick slices are prepared. Slices are cut using a 45-mm rotary blade, floated into ice cold oxygenated V-7 preservation buffer, and pre-incubated in the presence of serum free Williams Medium (WE) (available from Sigma Chemical Co., St. Louis, Mo.) containing D-glucose and gentamicin with 95% oxygen/5% CO₂ (carbogen) at 37 C for 30 min. Some slices may be taken at this time point and designated as Time 0 (to) slices. The rest of the slices are loaded onto titanium-screen rollers from Vitron, Inc. (Tucson, Ariz.) and inserted into sterile 20 ml glass vials containing 1.7 ml of WE media. The vials are capped with lids containing a 1 mm hole for the infusion of oxygen. This assembly is placed horizontally into a Dynamic Organ Culture incubator (available from Vitron Inc. (Tucson, Ariz.)) and incubated at 37 C in the presence of carbogen using a flow rate of 1.5 lpm.

Incubation of slices with ethanol is performed as follows. Following pre-incubation with WE, slices are incubated with media only (control), media+25 mM ethanol (ethanol), media+25 mM ethanol+0.50 mM 4-methylpyrazole as an optional positive control (ethanol +4-MP), media+0.50 mM 4-methylpyrazole (control+4-MP), control media+test compound, or ethanol media+test compound. The addition of 4-MP may be used in these studies as it is a general inhibitor of ethanol metabolism. The slices are placed in the Dynamic Organ Culture incubator and cultured at 37 C for up to 96 h, and every 24 h the appropriate media is replenished. In order to determine the concentration of ethanol and acetaldehyde in the media, the supernatant is analyzed using headspace gas chromatography.

Viability assays are performed as follows. Slice viability is determined by measuring adenosine triphosphate (ATP) and lactate dehydrogenase (LDH) levels. For the ATP assay, slices are harvested at the appropriate times, placed into 70% ethanol/2 mM EDTA, flash frozen, and stored at −70 C until assayed. Samples are thawed on ice, sonicated, and diluted in 0.1 M Tris-HCl/2 mM EDTA prior to use in a standard ATP assay kit.

For LDH determination, supernatant is collected and frozen at −70 C. The slice is solubilized in WE containing 2% Triton X-100 and LDH determined using a cytotoxicity detection kit (LDH) (e.g., available from Roche Applied Science, Penzberg, Germany). The absorbance of the samples is measured at 490 nm. All protein concentrations from the slices are determined using a BCA protein assay kit. To calculate the % Cytotoxicity at subsequent 24 h time points, the LDH in the media is divided by the total LDH in the PCLS and multiplied by 100.

ADH/ALDH activity is determined as follows. Slices are harvested at the time points indicated, washed in PBS (pH 7.4), lysed in 1% Triton X-100, and sonicated. For the ADH assay, protein concentrations are adjusted to 50-100 mg and incubated at 37 C in the presence of 10 mM ethanol, 3 mM NAD+, and 0.5 M Tris-HCl (pH 7.4). Conversion of NAD+ to NADH is measured by the change in optical density at 340 nm using a spectrophotometer. ALDH activity is determined by placing the slice into 1 ml of buffer containing 100 mM NaPO4 (pH 7.4), 3 mM NAD+, and 10 mM pyrazole. The reaction is initiated by adding propionaldehyde to a final concentration of 25 mM (low Km enzyme) or 1 mM (total enzyme activity). Conversion of NAD+ to NADH is determined by the change in optical density at 340 nm using a spectrophotometer.

Cytochrome P450 2E1 (CYP2E1) assay is performed as follows. Microsomes are prepared from slices using a modified known protocol. Briefly, slices are added to a 1.15% KCl solution, sonicated, subjected to differential centrifuged to obtain the microsomal fraction, and protein concentration determined. CYP2E1 activity is determined using the pnitrophenol (PNP) (Sigma Chemical Co., St. Louis, Mo.) oxidation assay described by Wu and Cederbaum (Wu, D., et al., Mol Pharmacol 1996; 49(5):802-7). Microsomal protein is added to 0.2 mM PNP, 1 mM NADPH (available, e.g., from Sigma Chemical Co., St. Louis, Mo.), and incubated at 37 C for 1 h. The reaction is stopped using 30% trichloroacetic acid, centrifuged, and ION NaOH added to the remaining supernatant. Activity is obtained by measuring the absorbance at 546 nm using a spectrophotometer. Immunoblotting techniques are used to determine microsomal CYP2E1 expression. Microsomal protein (5 mg) is loaded onto a 10% SDS polyacrylamide gel, transferred onto PVDF membrane, and blocked in Blotto. The primary antibody, rabbit anti-CYP2E1 (available from e.g., Chemicon, Temecula, Calif.) is incubated overnight at 4 C followed by 1 h incubation with an IR-labeled secondary anti-rabbit IgG antibody. Blots are washed, dried and scanned using an IR scanner. Densitometric analysis is performed using imaging software and the data are expressed in arbitrary densitometric units/mg of protein.

Cellular redox state and albumin secretion is measured as follows. Supernatant from liver slices incubated up to 96 h under various conditions is assayed for the presence of lactate or pyruvate using assay kits to assess the cellular redox state. Briefly, 50 ml/well of each sample is incubated with 50 ml/well of assay kit reagent in conjunction with the lactate or pyruvate enzyme. The reaction is allowed to take place for 30 min and the levels of lactate or pyruvate are determined by absorbance at 570 nm. Plates are analyzed using a plate reader. Supernatant is used to analyze PCLS for albumin secretion using a Rat Albumin Quantitative ELISA Kit. This assay is performed by coating a 96 well plate with a capture antibody (sheep anti-Rat Albumin). Plates are blocked with BSA, and the albumin standard or sample are added. A secondary antibody is added (HRP conjugated Sheep anti-Rat Albumin) and the plate is developed using TMB peroxidase substrate. Absorbance is detected at 450 nm using a plate reader.

Triglyceride analysis is performed as follows. At indicated time points, supernatant is removed and slices are washed in PBS (pH 7.4). Slices are placed in PBS containing 0.5% Triton-X100, sonicated, and the equivalent of 300 mg of protein assayed for triglycerides using the serum triglyceride kit. Triglycerides in each sample are hydrolyzed by lipase and the resulting free glycerol calculated against a glycerol standard at 540 nm using a spectrophotometer.

Oil Red O staining is performed as follows. For Oil Red O staining, slices are flash frozen in OCT (available, e.g., from Sigma Chemical Co., St. Louis, Mo.), sectioned and gently placed onto slides. Oil Red O is incubated with the sections, washed, and the presence or absence of fat content analyzed by light microscopy on a microscope by a pathologist.

Results

Outputs of this assay included:
Cytotoxcity (LDH and ATP)
Fats (Triglycerides)
Metabolism (Acetaldehyde)
Oxidative stress (MAA)
Cytokines (IL-6, MCP-1, TNF, IL-10, IL-1beta)

Triglycerides—Levels were significantly decreased in the I-1+ethanol PCLSs back to control levels.

Acetaldehyde—Levels in the I-1 treated slices appear to be decreased at all time points.

ATP—Levels remain constant at 24 and 48 hours. However, at 72 hours there is an increase with I-1 treatment.

Cytotoxicity—Levels were decreased in the ethanol treated and continued to drop with I-1 treatment.

The assay was not sensitive enough to measure reactivity of TNF-alpha, IL-10, and IL-1 beta.

Overall, the data indicated that compound I-1 acts as a chemoprotectant to reduce cellular stress and toxicity in response to ethanol treatment. These results are promising and support studies of compound I-1 in humans for treatment of alcohol induced hepatitis, fatty liver, and related diseases and disorders.

### Example 3: Compound I-1 in Various Hepatic Inflammation Models

We studied compound I-1 in additional preclinical models of inflammatory diseases, including:

Choline Deficient High Fat Diet Rat Study
Lipid profiles
Histopathology

STAM Mouse Model
Hepatic Fibrosis and Triglycerides
Body Weight Gain with High Fat Diet

NAFLD is diagnosed in the US as 3 million new cases each year. NASH is included in the NAFLD diagnosis, as it is the easiest to ascertain in a subject. NAFLD activity score (NAS) is scored as 0-2 not diagnostic, 3-4 ranges from not diagnostic/borderline/positive NASH, and 5-8 diagnostic of NASH; the NAS scoring system includes a composite of four semi-quantitative features: steatosis (0-3), lobular inflammation (0-2), hepatocellular ballooning (0-2), and fibrosis (0-4). To analyze the effect of compound I-1 on NAFLD, NAS was analyzed in rats treated with vehicle or various doses of compound I-1 (100 mg/kg, 125 mg/kg was compared to escalation 30/60/100 mg/kg and 50/75/100 mg/kg BID, respectively in animals fed a choline deficient, high fat diet for 7 weeks (vehicle vs. 100 mg/kg vs 30/60/100 mg/kg) and 12 weeks (vehicle vs. 125 mg/kg vs 50/75/100 mg/kg). Plasma was collected at 1, 2, 3, 6, 8, 10, and 12 weeks and tested for cytokines and ALT, AST, Triglycerides, Cholesterol, and tBilirubin (measures of liver function). At 12 weeks the livers were collected and gene expression studies, immunohistochemistry, and hydroxyproline. Over the 7 or 12 week period, food intake and weight gain did not change within these two comparison groups. In the 12 week group, there was a reduction in MIP expression levels compared to vehicle, MCP and Rantes expression levels remained similar to vehicle treated animals. Triglycerides and cholesterol trended at lower values than vehicle in both 7 week and 12 week groups in animals that received compound I-1. In histopathology of the collected livers, both the 7 week and 12 week compound I-1-treated animals trended to have reduced inflammation (hematoxylin eosin) and fibrosis (picrosirius red). The NAS score in the 7 week group demonstrated statistical significant reduction as compared to vehicle in the 100 mg/kg compound I-1 group, the 30/60/100 mg/kg escalation group was reduced.

In a STAM mouse model (NASH/HCC), animals at birth are given a low dose of STZ (200 ug) and then at 3 to 4 weeks these animals are fed a high fat diet. In the progression of the animal model, at 5 weeks fatty liver is evident, 7 weeks NASH is evident, 9 weeks fibrosis is evident, and at 10 weeks nodules are evident and necropsy is performed. In this study, three groups vehicle (0.5% methylcellulose) alone, BID (200 mg/kg in vehicle, BID=twice a day), and QID (200 mg/kg in vehicle, QID=three times a day), dosing began at 4 weeks and continued to week ten of the animal model. In both the BID and QID groups, there was a significant reduction of fibrosis and hepatic triglycerides. In both BID and QID compound I-1 treated groups there was a significant reduction in body weight gain as compared to the vehicle alone group.

We claim:

1. A method of treating a respiratory disease, disorder, or condition selected from chronic cough, atopic asthma, pneumonia, and pulmonary sepsis, or an organ disease, disorder, or condition selected from alcohol induced hepatitis, minimal change disease, and focal segmental glomerulosclerosis, comprising administering to a patient in need thereof an effective amount of a compound of Formula I: 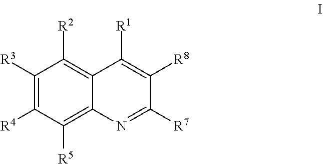
or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or 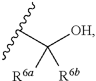
wherein one of R¹, R⁷, and R⁸ is —NH₂ and one of R¹, R⁷, and R⁸ is 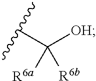
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

2. A method of treating a respiratory disease, disorder, or condition selected from chronic cough, atopic asthma, pneumonia, and pulmonary sepsis, or an organ disease, disorder, or condition selected from alcohol induced hepatitis, minimal change disease, and focal segmental glomerulosclerosis, comprising administering to a patient in need thereof an effective amount of a compound of Formula II: 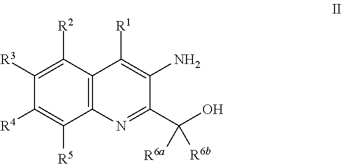
or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁-4 aliphatic optionally substituted with 1, 2, or 3 deuterium or halogen atoms; and
R^{6b} is C₁₋₄ aliphatic optionally substituted with 1, 2, or 3 deuterium or halogen atoms.

3. The method of claim 1 or 2, wherein R^{6a} and R^{6b} are methyl or ethyl optionally substituted with 1, 2, or 3 deuterium or halogen atoms.

4. The method of any one of claims 1 to 3, wherein R^{6a} and R^{6b} are methyl.

5. The method of claim 1 or 2, wherein the compound is selected from: 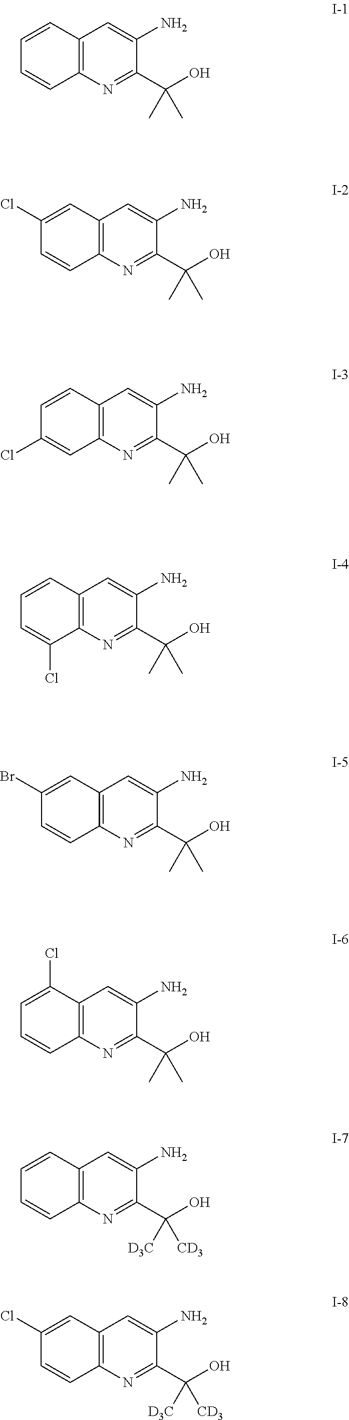 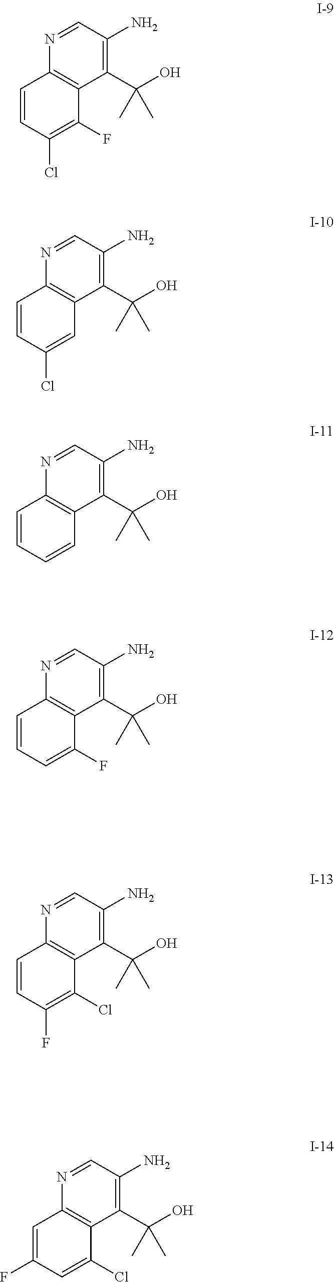 [SMILES: CC(C)(O)c1c(N)cnc2ccc(Cl)c(F)c12 ; CC(C)(O)c1c(N)cnc2ccc(Cl)cc12 ; CC(C)(O)c1c(N)cnc2ccccc12 ; CC(C)(O)c1c(N)cnc2cccc(F)c12 ; CC(C)(O)c1c(N)cnc2ccc(F)c(Cl)c12 ; CC(C)(O)c1c(N)cnc2cc(F)cc(Cl)c12] 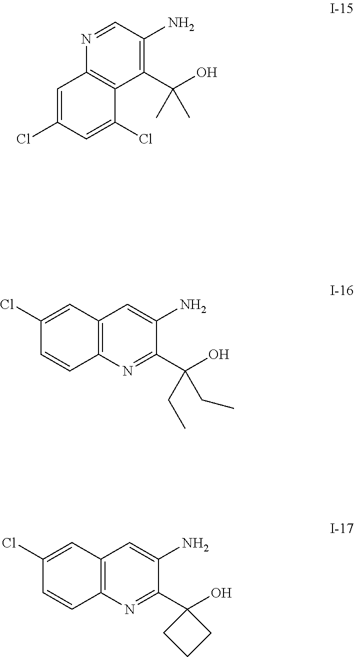 [SMILES: CC(C)(O)c1c(N)cnc2cc(Cl)cc(Cl)c12 ; CCC(O)(CC)c1nc2ccc(Cl)cc2cc1N ; Nc1cc2cc(Cl)ccc2nc1C1(O)CCC1] 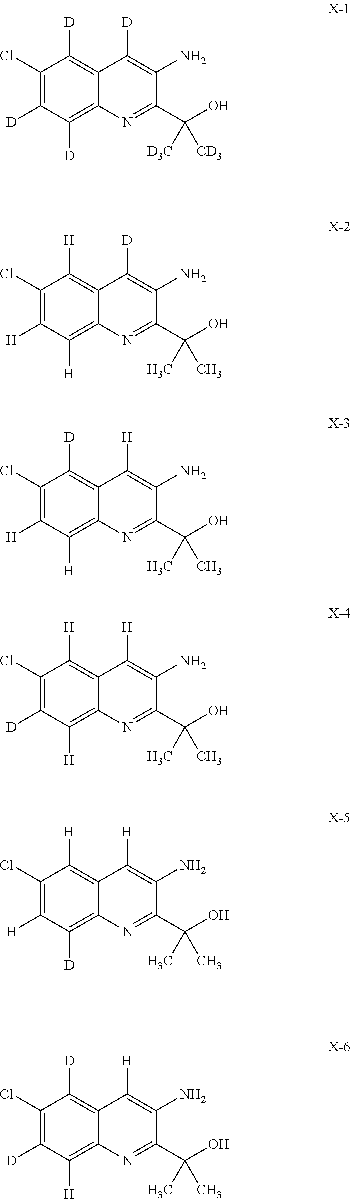 [SMILES: [2H]c1c(Cl)c([2H])c2c([2H])c(N)c(C(O)(C([2H])([2H])[2H])C([2H])([2H])[2H])nc2c1[2H] ; [2H]c1c(N)c(C(C)(C)O)nc2ccc(Cl)cc12 ; [2H]c1c(Cl)ccc2nc(C(C)(C)O)c(N)cc12 ; [2H]c1cc2nc(C(C)(C)O)c(N)cc2cc1Cl ; [2H]c1cc(Cl)cc2cc(N)c(C(C)(C)O)nc12 ; [2H]c1cc2nc(C(C)(C)O)c(N)cc2c([2H])c1Cl] 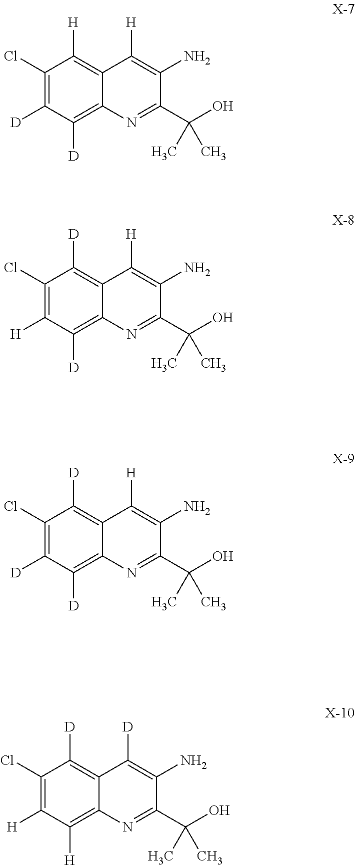 [SMILES: [2H]c1c(Cl)cc2cc(N)c(C(C)(C)O)nc2c1[2H] ; [2H]c1c(Cl)cc([2H])c2nc(C(C)(C)O)c(N)cc12 ; [2H]c1c(Cl)c([2H])c2cc(N)c(C(C)(C)O)nc2c1[2H] ; [2H]c1c(Cl)ccc2nc(C(C)(C)O)c(N)c([2H])c12] 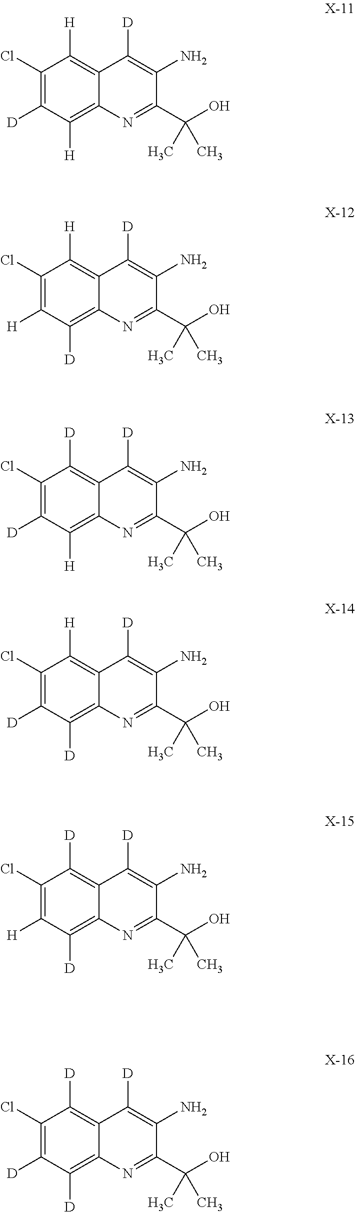 [SMILES: [2H]c1cc2nc(C(C)(C)O)c(N)c([2H])c2cc1Cl ; [2H]c1c(N)c(C(C)(C)O)nc2c([2H])cc(Cl)cc12 ; [2H]c1cc2nc(C(C)(C)O)c(N)c([2H])c2c([2H])c1Cl ; [2H]c1c(Cl)cc2c([2H])c(N)c(C(C)(C)O)nc2c1[2H] ; [2H]c1cc(Cl)c([2H])c2c([2H])c(N)c(C(C)(C)O)nc12 ; [2H]c1c(Cl)c([2H])c2c([2H])c(N)c(C(C)(C)O)nc2c1[2H]] 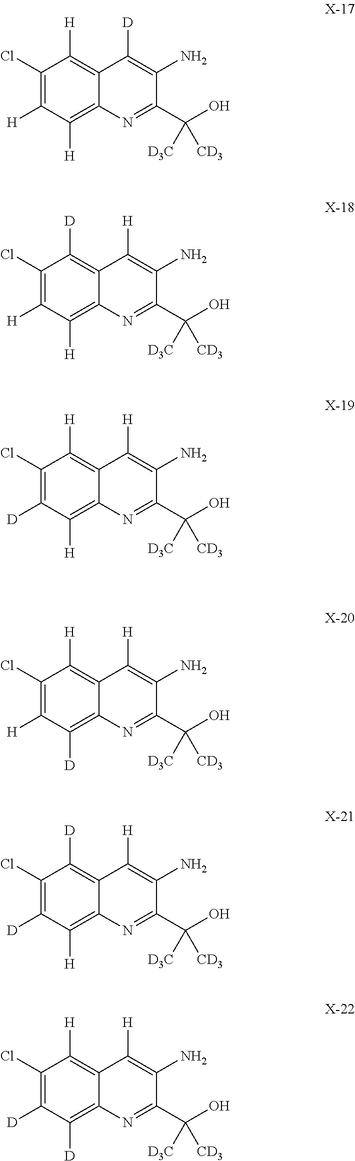 [SMILES: [2H]c1c(N)c(C(O)(C([2H])([2H])[2H])C([2H])([2H])[2H])nc2ccc(Cl)cc12 ; [2H]c1c(Cl)ccc2nc(C(O)(C([2H])([2H])[2H])C([2H])([2H])[2H])c(N)cc12 ; [2H]c1cc2nc(C(O)(C([2H])([2H])[2H])C([2H])([2H])[2H])c(N)cc2cc1Cl ; [2H]c1cc(Cl)cc2cc(N)c(C(O)(C([2H])([2H])[2H])C([2H])([2H])[2H])nc12 ; [2H]c1cc2nc(C(O)(C([2H])([2H])[2H])C([2H])([2H])[2H])c(N)cc2c([2H])c1Cl ; [2H]c1c(Cl)cc2cc(N)c(C(O)(C([2H])([2H])[2H])C([2H])([2H])[2H])nc2c1[2H]] 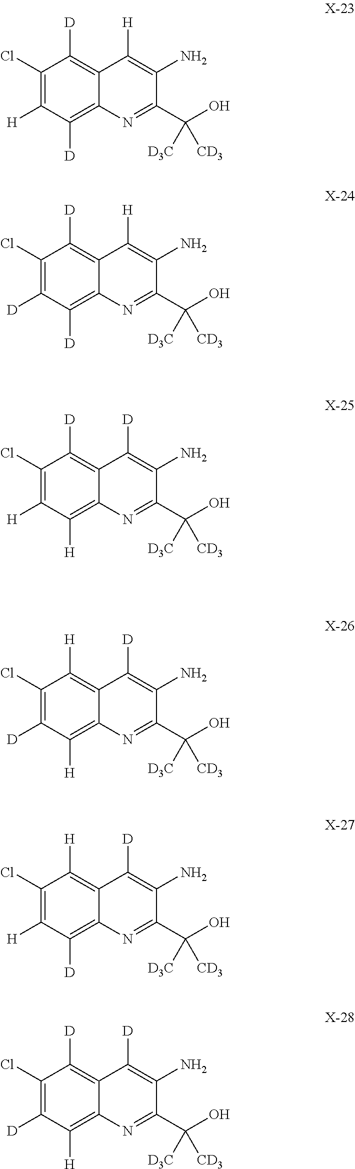 [SMILES: [2H]c1c(Cl)cc([2H])c2nc(C(O)(C([2H])([2H])[2H])C([2H])([2H])[2H])c(N)cc12 ; [2H]c1c(Cl)c([2H])c2cc(N)c(C(O)(C([2H])([2H])[2H])C([2H])([2H])[2H])nc2c1[2H] ; [2H]c1c(Cl)ccc2nc(C(O)(C([2H])([2H])[2H])C([2H])([2H])[2H])c(N)c([2H])c12 ; [2H]c1cc2nc(C(O)(C([2H])([2H])[2H])C([2H])([2H])[2H])c(N)c([2H])c2cc1Cl ; [2H]c1c(N)c(C(O)(C([2H])([2H])[2H])C([2H])([2H])[2H])nc2c([2H])cc(Cl)cc12 ; [2H]c1cc2nc(C(O)(C([2H])([2H])[2H])C([2H])([2H])[2H])c(N)c([2H])c2c([2H])c1Cl] 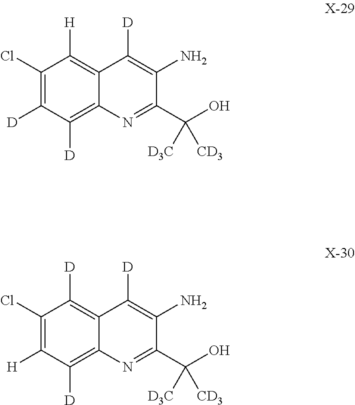 [SMILES: [2H]c1c(Cl)cc2c([2H])c(N)c(C(O)(C([2H])([2H])[2H])C([2H])([2H])[2H])nc2c1[2H] ; [2H]c1cc(Cl)c([2H])c2c([2H])c(N)c(C(O)(C([2H])([2H])[2H])C([2H])([2H])[2H])nc12]
or a pharmaceutically acceptable salt thereof.

6. The method of claim 1 or 2, wherein the compound is 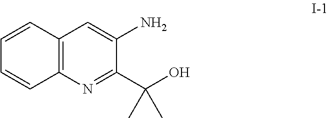
or a pharmaceutically acceptable salt thereof.

7. The method of claim 1 or 2, wherein the compound is 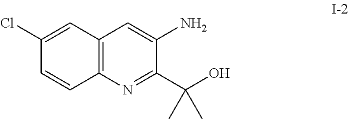 [SMILES: CC(C)(O)c1nc2ccc(Cl)cc2cc1N]
or a pharmaceutically acceptable salt thereof.

8. The method of any one of claims 1 to 7, wherein the treatment is for chronic cough.

9. The method of claim 0, wherein the chronic cough is associated with upper airway cough syndrome.

10. The method of claim 0, wherein the chronic cough is associated with gastroesophageal reflux disease or laryngopharyngeal reflux disease.

11. The method of claim 0, wherein the chronic cough is associated with asthma.

12. The method of claim 0, wherein the chronic cough is associated with non-asthmatic eosinophilic bronchitis.

13. The method of claim 0, wherein the patient has a history of one or more of the following: treatment with angiotensin-converting enzyme (ACE) inhibitor, smoking, asthma, exposure to environmental respiratory irritants, and bronchitis.

14. The method of any one of claims 1 to 7, wherein the treatment is for pneumonia, wherein the pneumonia is not associated or concurrent with acute respiratory distress syndrome (ARDS).

15. The method of any one of claims 1 to 7, wherein the treatment is for pneumonia that has a differential diagnosis from eosinophilic pneumonia.

16. The method of claim 14 or 15, wherein the pneumonia is community-acquired pneumonia.

17. The method of claim 14 or 15, wherein the pneumonia is nocosomial pneumonia.

18. The method of claim 14 or 15, wherein the pneumonia is bacterial pneumonia or viral pneumonia.

19. The method of claim 18, wherein the patient is diagnosed with a bacterial infection by Streptococcus pneumoniae, Haemophilus influenzae, S. aureus, Group A streptococci, Moraxella catarrhalis, Klebsiella pneumoniae, Pseudomonas aeruginosa, Legionella spp, Mycoplasma pneumoniae, Chlamydia pneumoniae, or C. psittaci.

20. The method of claim 18, wherein the patient is diagnosed with a viral infection by influenza virus, respiratory syncytial virus (RSV), parainfluenza, metapneumovirus, coronavirus, rhinovirus, hantavirus, or adenovirus.

21. The method of any one of claims 14 to 20, wherein the pneumonia treated is lobar pneumonia.

22. The method of any one of claims 14 to 21, wherein the pneumonia treated is upper, middle or lower lobe pneumonia.

23. The method of any one of claims 14 to 20, wherein the pneumonia treated is focal pneumonia, alveolar pneumonia, or interstitial pneumonia.

24. The method of any one of claims 14 to 20, wherein the pneumonia treated is bronchial pneumonia.

25. The method of any one of claims 1 to 7, wherein the treatment is for pulmonary sepsis or sepsis induced lung injury.

26. The method of claim 25, wherein the pulmonary sepsis or sepsis induced lung injury is without acute respiratory distress syndrome (ARDS).

27. The method of any one of claims 1 to 7, wherein the treatment is for alcohol induced hepatitis.

28. The method of claim 27, wherein the alcohol induced hepatitis treated is without cirrhosis.

29. The method of claim 27, wherein the patient with alcohol induced hepatitis is determined to have elevated levels of aspartate aminotransferase (AST) and/or alanine aminotransferase (ALT) as compared to levels in a control group not afflicted with alcohol induced hepatitis.

30. The method of claim 29, wherein the levels of AST in the control group is about 8 to 48 IU/L, and the levels of ALT in the control group is about 7 to 55 IU/L.

31. The method of claim 29, wherein the patient is determined to have an AST:ALT ratio of greater than 2:1.

32. The method of any one of claims 1 to 7, wherein the treatment is for minimal change disease.

33. The method of claim 32, wherein the minimal change disease treated is associated with nephrotic syndrome.

34. The method of claim 32 or 33, wherein the minimal change disease treated is concurrent with proteinuria.

35. The method of any one of claims 1 to 7, wherein the treatment is for focal segmental glomerulosclerosis (FSGS).

36. The method of claim 35, wherein the FSGS treated is primary FSGS.

37. The method of claim 35, wherein the FSGS treated is secondary FSGS.

38. The method of claim 35, wherein the FSGS treated is familial FSGS.

39. The method of any one of claims 35 to 38, wherein the FSGS treated is associated with nephrotic syndrome.

40. The method of any one of claims 35 to 38, wherein the FSGS treated is concurrent with kidney failure or proteinuria.

41. The method of any one of claims 35 to 40, wherein the patient with FSGS has a prior history of minimal change disease.

42. The method of any one of claims 1 to 41, wherein the compound or pharmaceutically acceptable salt thereof is administered systemically.

43. The method of any one of claims 1 to 42, wherein the compound or pharmaceutically acceptable salt thereof is administered orally.

44. The method of any one of claims 1 to 43, wherein the compound or pharmaceutically acceptable salt thereof is administered at a dose of about 10 mg to about 7500 mg per day.

45. The method of any one of claims 1 to 43, wherein the compound or pharmaceutically acceptable salt thereof is administered at a dose of about 50 mg to about 3600 mg per day.

46. The method of any one of claims 1 to 43, wherein the compound or pharmaceutically acceptable salt thereof is administered at a dose of about 250 mg to about 2400 mg per day.
